(19) United States
(12) Patent Application Publication
(10) Pub. No.: US 2022/0040065 A1
(43) Pub. Date: Feb. 10, 2022

(54) COMPOSITIONS AND METHODS FOR PREVENTION OF VIRAL INFECTIONS AND ASSOCIATED DISEASES

(71) Applicant: Micropure, Inc., Scottsdale, AZ (US)

(72) Inventors: Jaiprakash G. Shewale, Scottsdale, AZ (US); James L. Ratcliff, Scottsdale, AZ (US)

(73) Assignee: Micropure, Inc., Scottsdale, AZ (US)

(21) Appl. No.: 17/377,103

(22) Filed: Jul. 15, 2021

Related U.S. Application Data
(63) Continuation of application No. PCT/US21/41682, filed on Jul. 14, 2021.
(60) Provisional application No. 63/061,019, filed on Aug. 4, 2020.
(60) Provisional application No. 63/071,589, filed on Aug. 28, 2020.
(60) Provisional application No. 63/089,474, filed on Oct. 8, 2020.
(60) Provisional application No. 63/110,809, filed on Nov. 6, 2020.
(60) Provisional application No. 63/142,788, filed on Jan. 28, 2021.
(60) Provisional application No. 63/177,821, filed on Apr. 21, 2021.

(51) Int. Cl. A61K 8/20; A61Q 11/00; A61Q 17/00

(52) U.S. Cl. CPC A61K 8/20; A61Q 17/005; A61Q 11/00

(57) ABSTRACT

Described herein are single-phase multi-component compositions and methods for their use reducing the viral load of infectious viruses on host organisms, including but not limited to Influenza A, Rhinovirus Type 14, Adenovirus Type 5, Herpes Simplex Virus Type 1, Herpes Simplex Virus Type 2, Severe Acute Respiratory Syndrome Coronavirus-2 (SARS-CoV-2; COVID-19 virus), Severe Acute Respiratory Syndrome Coronavirus (SARS-CoV), and Human coronavirus 229E. The compositions described herein comprise a combination of an oxidative compound and a buffering system. The methods of use of these compositions as antiviral agents when applied to the oral, nasal, ocular, aural, anal, and urogenital cavities are described. Data shows the compositions to be effective in reducing influenza A and the human coronaviruses. In that regard, the single-phase multi-component compositions and methods for its antiviral use disclosed herein may be offer a single, effective product to combat multiple, concurrent pandemics.

### CROSS REFERENCE TO RELATED APPLICATIONS

This non-provisional application is a continuation of PCT Patent Application No. PCT/US21/41682, entitled “COMPOSITIONS AND METHODS FOR PREVENTION OF VIRAL INFECTIONS AND ASSOCIATED DISEASES,” filed on Jul. 14, 2021, and claims priority to, and the benefit of U.S. Provisional Application No. 63/061,019, entitled “COMPOSITIONS AND METHODS FOR PREVENTION OF VIRAL INFECTIONS AND ASSOCIATED DISEASES,” filed on Aug. 4, 2020, U.S. Provisional Application No. 63/071,589, entitled “COMPOSITIONS AND METHODS FOR PREVENTION OF VIRAL INFECTIONS AND ASSOCIATED DISEASES,” filed on Aug. 28, 2020, U.S. Provisional Application No. 63/089,474, entitled “COMPOSITIONS AND METHODS FOR PREVENTION OF VIRAL INFECTIONS AND ASSOCIATED DISEASES,” filed on Oct. 8, 2020, U.S. Provisional Application No. 63/110,809, entitled “COMPOSITIONS AND METHODS FOR PREVENTION OF VIRAL INFECTIONS AND ASSOCIATED DISEASES,” filed on Nov. 6, 2020, US Provisional Application No. 63/142,788, entitled “COMPOSITIONS AND METHODS FOR PREVENTION OF VIRAL INFECTIONS AND ASSOCIATED DISEASES,” filed on Jan. 28, 2021, and U.S. Provisional Application No. 63/177,821, entitled “COMPOSITIONS AND METHODS FOR PREVENTION OF VIRAL INFECTIONS AND ASSOCIATED DISEASES,” filed on Apr. 21, 2021, all of which are hereby incorporated by reference in their entirety.

### TECHNICAL FIELD

The present disclosure relates generally to a single-phase multi-component composition for the disinfection of the oral, nasal, ocular, and aural cavities of humans and animals and for the reduction, deactivation, or reducing infectivity of pathogenic viruses such as Influenza A, human coronaviruses, Rhinovirus Type 14, Adenovirus Type 5, Herpes Simplex Virus Type 1 and Herpes Simplex Virus Type 2 in those cavities. Examples of human coronaviruses include but not limited to Middle East Respiratory Syndrome virus (MERS-CoV), Severe Acute Respiratory Syndrome virus (SARS-CoV or SARS virus), Coronavirus disease-19 virus (SARS-CoV-2, COVID-19 virus or 2019-nCoV), and human coronavirus strain 229 E (HCoV-229E).

### BACKGROUND

Infectious diseases resulting from viruses are highly contagious. Further, viral infection leads to other complex clinical diseases such as acute respiratory distress syndrome, pneumonia, heart failure, blood clotting disorders, multisystem inflammatory syndrome (MIS-C), renal failure, liver damage, shock and multi-organ failure.

Example of viruses causing infections include adenovirus, canine distemper virus, cytomegalovirus, Epstein-Barr virus, human papillomavirus, feline calicivirus, herpesvirus, rhinovirus, human immunodeficiency virus (HIV), parvovirus, measles virus, polio virus, rotavirus, Influenza A (Flu A), Middle East Respiratory Syndrome virus (MERS-CoV or MERS virus), Severe Acute Respiratory Syndrome virus (SARS-CoV or SARS virus), Coronavirus disease-19 virus (SARS-CoV-2, COVID-19 virus or 2019-nCoV), and human coronavirus strain 229E (HCoV-229E). SARS-CoV, SARS-CoV-2, Influenza A, Rhinovirus, Adenovirus, Herpes Simplex Virus are specific and significant threats, in particular to oral and nasopharynx health.

Contagious viruses are those that transmit from human to human, including but not limited to HIV, Influenza A, MERS-CoV, SARS-CoV, SARS-CoV-2, and HCoV-229E.

Viruses have some common structural elements. Ribonucleic acid (RNA) or deoxyribonucleic acid (DNA) molecules are encapsulated within a complex protein structure. Some viruses comprise lipids in conjunction with proteins to form the envelope. The outer macromolecular cover protects RNA or DNA from degradation and has binding sites that bind to the receptors on host cells and thereby cause infection.

The highly pathogenic viruses MERS-CoV (MERS virus), SARS-CoV (SARS virus), and SARS-CoV-2 (COVID-19 virus) are members of the Coronavirus family.

Coronaviruses are a group of RNA viruses that cause diseases in mammals and birds. These viruses have characteristic club-shaped spikes that project from their surface, which in electron micrographs create an image reminiscent of the solar corona, hence the name coronavirus. They are positive-sense single-stranded RNA viruses. There are four main sub-groupings of coronaviruses, known as alpha, beta, gamma, and delta. Hundreds of coronaviruses exist, most of which circulate in animals like pigs, camels, bats and cats (see www.niaid.nih .gov/diseases-conditions/coronaviruses).

The seven strains of coronaviruses that are known to infect people include HCoV-229E, HCoV-NL63, HCoV-0C43, HCoV-HKU1, SARS-CoV, MERS-CoV, and SARS-CoV-2 (see www.cdc.gov/coronavirus/types.html), Chen, B., Tian, E., He, B. et al. Sig Transduct Target Ther, Vol. 5, p. 89 (2020) (see doi. org/10.1038/s41392-020-0190-2).

People around the world commonly get infected with human coronavirus strains 229E, NL63, 0C43, and HKU1 that cause common cold symptoms and are responsible for approximately one-third of the cases of the common cold. (Chen, B., Tian, E., He, B. et al. Sig Transduct Target Ther Vol 5, p. 89 (2020); Sampson, V., Kamona, N., and Sampson, A., British Dental J, Vol 228, p. 971 (2020); Gaunt, E. R., Hardie, A., Claas, E. C. et. al., Journal of Clinical Microbiology, Vol 48, p. 2940 (2010); Lim, Y., Ng, Y., Tam, J., et. al. Diseases, Vol 4, p. 26 (2016)). Unlike MERS-CoV, SARS-CoV, and SARS-CoV-2, these infections do not lead to high rates of permanent organ damage and death. Permanent organ damage frequently includes damage to the respiratory system, septic shock, acute kidney injury, cardiac injury and lymphocytopenia (Yu, Y., Xu, D, Fu, S. et al, Crit Care, Vol 24, p 219, (2020).

Influenza A was first detected in humans in 2011. It is part of the Orthomyxovirus family, including Influenza A, B, C, Thogotovirus and Isavirus. Influenza A and SARS CoV-2 have similar methods of transmission, infection and clinical manifestation. The Hong Kong Flu pandemic (1968-89) resulted from the H3N2 variant of Influenza A, which descended from the H2N2 variant by an antigenic shift wherein genes from multiple subtypes reassort to form a new virus variant. (Zayet, Souheil et al. Clinical features of COVID-19 and Influenza Microbes and Infection, 22 (2020) 481-488. DOI: 10.1016/j.micinf.2020.05.016; Zhang, Naru et al. Recent advances in the detection of respiratory virus infection in humans. J of Medical Virology, 92(4), 2020, 408-417. doi: 10.1002/jmv.25674. Epub 2020 Feb 4 DOI: 10.1002/jmv.25674).

The H3N2 and H1N1 Influenza A viruses are still circulating in the human population. Because of its host-range diversity, genetic and antigenic diversity, and its ability to reassort generally, Influenza A is considered likely to return in the form of a future pandemic. (Mostafa, Ahmed et al. Zoonotic Potential of Influenza A Viruses: A Comprehensive Overview, Viruses 10(9) 2018: 419. Published online 2018 Sep 13. doi: 10.3390/v10090497. Taubenberger, Jeffery and Morens, David. The Pathology of Influenza Virus Infections. Annu Review Pathology, 2008, 499-522. DOI: 10.1146/annurev.pathmechdis.3.121806.154316).

Major routes of entry of contagious viruses like Influenza A, MERS-CoV, SARS-CoV, and SARS-CoV-2 into the human body are through the nasal channel, mouth, and eyes. Contact touching plays a key role in the spread of such infectious diseases as viruses deposited on hands after touching a contaminated surface may get into the human body after touching the face particularly in close proximity to the mouth, nose and eyes. Transmission of coronaviruses is believed to occur when respiratory aerosol droplets are generated by sneezing, coughing, breathing and talking. Viral load is important in transmission. It is thought that a higher viral load of SARS-CoV-2 in the oral cavity or saliva increases the risk of its transmission from one individual to another during ordinary conversations. In addition, viral load is related to severity of infection and mortality rates. Thus, disinfection of the oral and nasal cavities may be an important factor for slowing transmission. SARS CoV-2 has been detected in the nasopharynx, oropharynx and in human saliva (Yoon, J. G., Yoon, J, Song, J.Y. et al J Korean Med Sci, Vol. 35, p.1 (2020).

The infection or entry of a virus into a host cell is mediated by a receptor on the host cell surface. That receptor is recognized by viral surface protein and/or lipid molecules. For highly infectious viruses such as SARS-CoV and SARS-CoV-2, the host cell receptor in tissues present in the oral cavity such as oral mucosa, tongue, salivary glands, and throat has been identified as the angiotensin-converting enzyme II (ACE-2) (Xu H. et.al., International Journal of Oral Science, Vol. 12:8, www.nature. com/articles/s41368-020-0075-9.pdf; Yao Y. Journal of Dental Research, Apr. 9, 2020, journals.sagepub.com/doi/10.1177/0022034520918518).

Viral infections that target the respiratory system often lead to pneumonia. Examples of such viruses are Influenza A, MERS-CoV, SARS-CoV, and SARS-CoV-2. Some of these viral infections may lead to acute respiratory syndrome (ARDS). (Dhama K. et al. Clin Microbiol Rev, Vol. 23(4) e00028-20. https://doi. org/10.1128/CMR.00028-20. 2020).

There is a dearth of information relating to reduction of MERS-CoV, SARS-CoV, and SARS-CoV-2 viruses in the oral cavity and nasal channel as a means of preventing or reducing transmission of SARS-CoV-2 from an infected individual to another individual. Human saliva, in particular, may play a pivotal role in human-to-human transmission of COVID-19, and disinfecting or reducing the viral load in the oral cavity and saliva may have a significant role in reducing transmission of the disease (Sabino-Silva, R., Jardim, A. C. G., Siqueria, W. L. Clin Oral Invest, Vol 20, p. 1619 (2020).

Coronaviruses are spherical or pleomorphic enveloped particles containing single-stranded RNA associated with a nucleoprotein within a capsid comprised of matrix protein. The envelope bears club-shaped glycoprotein projections termed spike proteins. The viral genomic RNA is associated with the nucleocapsid phosphoprotein (N) and the viral envelope is made up of a lipid bilayer and structural proteins termed membrane (M), envelope (E) and spike (S) proteins (Attica I.M. et al. Molecular biology of coronaviruses: current knowledge; Heliyon 6 (2020) e04743).

Protein structure and functional relationship of highly pathogenic MERS-CoV, SARS-CoV and SARS-CoV-2 is will understood. N protein is structurally bound to the nucleic acid material of the virus forming the helical ribonucleoprotein, S protein (also known as spike protein) forms homotrimers and protrude in the viral surface facilitate binding of the virus to ACE-2 receptor enabling its entry into host cells thereby infecting the host cells, M protein is the most tightly structured protein that determines the shape of the virus, and E protein plays a role in production and maturation of the virus (Hatmal M. M. et. al. Comprehensive Structural and Molecular Comparison of Spike Proteins of SARS-CoV-2, SARS-CoV and MERS-CoV, and Their Interactions with ACE2; Cells 9, 2638 (2020); doi:10.3390/ cells9122638).

Of the four structural proteins, S protein is a prime target for reducing the infectivity of the virus as it is exposed and binds to the ACE-2 receptor for its entry into the host cell. Therefore, certain changes to the structure of this protein such as binding of antibody, chemical modification, disruption of its tertiary (three-dimensional) structure, and degradation that inhibits its binding to the ACE-2 receptor would result in reduced infectivity. The primary structure of S proteins in MERS-CoV, SARS-CoV and SARS-CoV-2 are not identical though some regions are conserved.

Deaths from COVID-19 are often accompanied by comorbidity factors, particularly Influenza and pneumonia. For example, of the 181,106 deaths in the United States attributed to COVID-19 during the period of Feb. 1, 2020 to Sep. 21, 2020, 80,088 (approximately 44.2%) involved comorbidity of Influenza A and another pathogen that causes pneumonia. (see www.cdc. gov/nchs/nvss/vsrr/covid weekly/index.htm, accessed Sep. 29, 2020). While a number of respiratory and circulatory diseases exhibit comorbidity with COVID-19 along with other compromising factors, influenza and pneumonia are most prevalent.

A twin pandemic may occur when there is widespread simultaneous infections by two contagious viruses, such as SARS-CoV-2 and Influenza A virus. Therefore, it is desirable that a composition reduces viral load or infectivity of multiple infectious viruses. It is further desirable that a composition that reduces the viral load of SARS-CoV-2 also reduces the viral load of influenza and the bacterial load of bacterial pneumonia.

Seasonal respiratory illnesses peak in fall and winter seasons of the temperate regions and are associated with spikes in the number of visits to the doctor's office and to hospital admissions (Thompson WW. Journal of Infectious Diseases. 2006;(Supplement 2): S82-91. doi: 10.1086/ 507558). While numerous respiratory pathogens are associated with hospitalization, influenza, human metapneumovirus, respiratory syncytial virus, rhinovirus, and parainfluenza virus are predominant, all of which cause similar symptoms (Gilca R, et al. Open Forum Infectious Diseases. 2014;1:ofu086. doi: 10.1093/ ofid/ofu086). Importantly, influenza-associated illness represents a significant proportion of these medical events. Influenza-related severe outcomes, such as death, ICU admission, or the need for invasive mechanical ventilation, for the most part befall elderly individuals or individuals with numerous comorbidities; however, previously healthy adults are also at risk for serious illness from influenza (Puig-Barbera J, BMC Public Health. 2014; Vol.14, p.564. doi: 10.1186/ 1471-2458-14-564). Influenza is a viral pathogen that causes an estimated 250,000 to 500,000 deaths annually (see www. who. int/en/news-room/detail/14-12-2017-up-to-650-000-people-die-of-respiratory-diseases-linked-to-seasonal-flu-each-year). These deaths may be directly related to influenza or related comorbidity factors.

There are four types of influenza viruses: A, B, C and D. Human influenza A and B viruses cause seasonal epidemics of disease (commonly known as the “flu season”) almost every winter in the United States. However, influenza A viruses are the only influenza viruses known to cause flu pandemics, i.e., global epidemics of flu disease (see www. cdc. gov/flu/about/viruses/types.htm, accessed Sep. 29, 2020). A pandemic can occur when a variation or mutation of influenza A virus emerges that infects large numbers of people and spreads quickly between people. Influenza type C infections generally cause mild illness and are not thought to cause human flu epidemics. Influenza D viruses primarily affect cattle and are not known to infect or cause illness in people. Influenza A viruses are RNA viruses. The core RNA structure is covered with a lipid membrane complexed with hemagglutinin and the neuraminidase glycoproteins in a ratio of approximately four to one (Bouvier N. M. and Palese P. Vaccine, Vol. 26(Suppl 4), pp. D49-D53, 2008; www.ncbi.nlm.nih.gov/pmc/articles/PMC3074182/). Hemagglutinin of the Influenza A virus binds to sialic acids on the cells of the respiratory tract for entry of the virus and infection of the cells (Ramos I. and Fernandez-Sesma A., Frontiers in Microbiology, Vol.3, Article 117, 2012; doi: 10.3389/ fmicb.2012.00117).

Rhinovirus is a common viral infectious agent in humans and is the predominant cause of the common cold; up to 80% of common cold illnesses may be associated with the rhinovirus infection during early fall and spring, Symptoms include sore throat, runny nose, nasal congestion, sneezing and cough. Symptoms like muscle aches, fatigue, malaise, headache or loss of appetite are sometimes observed. In addition to these upper respiratory tract syndromes, rhinovirus infection has also been associated with lower respiratory tract symptoms. it is widely accepted in the scientific literature that rhinovirus exacerbates asthma in school-aged children (Turner R. B., Rhinovirus: More than Just a Common Cold Virus, The Journal of Infectious Diseases, Vol. 195, Pages 765-766, (2007) https: //doi. org/10.1086/511829).

Adenoviruses can cause a wide range of illnesses with common cold or flu-like symptoms, fever, sore throat, acute bronchitis, pneumonia, pink eye, and acute gastroenteritis. People with weakened immune systems, or existing respiratory or cardiac disease, are at higher risk of developing severe illness from an adenovirus infection (see https: //www.cdc. gov/adenovirus/about/symptoms.html, accessed Apr. 9, 2021). In 2011, an adenovirus-related acute respiratory disease in Tayside, United Kingdom led to a case mortality rate of 23%. More than 100 serologically distinct types of adenovirus have been identified, including about 50 types that infect humans. Adenovirus Type 5 is the most studied type. There is a lack of suitable anti-adenoviral therapy for many immune-compromised patients. Human adenovirus infects the mucosa of the respiratory, gastrointestinal, and urogenital tracts as well as the eye. There is no FDA approved treatment of adenovirus infection. (Hoffman M. et al. Adv Concepts in Human Immunology: Prospects for Disease Control, 2020. Doi: 10.1007/ 978-3-030-33946-3_1).

Infection with herpes simplex virus, commonly known as herpes, can be due to either herpes simplex virus type I (HSV-1) or herpes simplex virus type 2 (HSV-2). HSV-1 is mainly transmitted by oral-to-oral contact to cause infection in or around the mouth (oral herpes). However, HSV-1 can also be transmitted through oral-genital contact to cause infection in or around the genital area (genital herpes). HSV-2 is almost exclusively transmitted through genital-to-genital contact during sex, causing infection in the genital or anal area (genital herpes). Both oral herpes infections and genital herpes infections are mostly asymptomatic and often go unrecognized but also can cause symptoms of painful blisters or ulcers at the site of infection, ranging from mild to severe. Most HSV-1 infections are acquired during childhood and infection is lifelong. In 2016, an estimated 3.7 billion people under the age of 50 had HSV-1 infection. HSV-2 infection is almost exclusively sexually transmitted, causing genital herpes. HSV-2 is the main cause of genital herpes. Infection with HSV-2 is lifelong and incurable. In 2016, an estimated 491 million people aged 15 to 49 years worldwide were flying with the genital herpes caused by HSV-2. More women are infected with HSV-2. than men as sexual transmission of HSV is more efficient from men to women than from women to men (see https:www.who.int/news-room/fact-sheets/detail/herpes-simplex-virus; accessed on Apr. 9, 2021).

Oxidative compounds may be prone to degradation in composition. For example, oxidative compounds may react chemically, such as with the hydroxy groups of polyhydroxy alcohols, leading to consumption of the oxidative compounds or active ingredients in compositions, thereby making the formulation ineffective or reducing the shelf life of the product. Accordingly, various challenges confront the manufacture of therapeutic and cosmetic pharmaceutical products containing oxidative compounds.

### SUMMARY

In various embodiments, a method is provided herein for reducing an initial viral load of Severe Acute Respiratory Syndrome Coronavirus-2 (SARS-CoV-2), comprising obtaining a single-phase multi-component antiviral composition comprising from about 0.0005% to about 5.0% of at least one of sodium chlorite or potassium chlorite, based on a total weight of the single-phase multi-component antiviral composition, a buffering system, wherein pH of the single-phase multi-component antiviral composition is between 6.0 and 8.0, and water, and contacting the single-phase multi-component antiviral composition with SARS-CoV-2 in a suspension for a contact time of at least 30 seconds, thereby reducing the initial viral load of SARS-CoV-2 by at least 98.4%.

A method for reducing an initial viral load of Influenza A, comprising obtaining a single-phase multi-component antiviral composition comprising from about 0.0005% to about 5.0% of at least one of sodium chlorite or potassium chlorite, based on a total weight of the single-phase multi-component antiviral composition, a buffering system, wherein pH of the single-phase multi-component antiviral composition is between 6.0 and 8.0, and water; and contacting the single-phase multi-component antiviral composition with Influenza A in a suspension for a contact time of at least 30 seconds, thereby reducing the initial viral load of Influenza A by at least 99.9%.

A method for reducing an initial viral load of human coronavirus strain 229E (HCoV-229E), comprising obtaining a single-phase multi-component antiviral composition comprising from about 0.0005% to about 5.0% of at least one of sodium chlorite or potassium chlorite, based on a total weight of the single-phase multi-component antiviral composition, a buffering system, wherein pH of the single-phase multi-component antiviral composition is between 6.0 and 8.0, and water; and contacting the single-phase multi-component antiviral composition with HCoV-229E in a suspension for a contact time of at least 30 seconds, thereby reducing the initial viral load of HCoV-229E by at least 82.2%.

A method for reducing an initial viral load of a virus, the virus comprising at least one of Herpes Simplex Virus Type 1, Herpes Simplex Virus Type 2, Rhinovirus Type 14 or Adenovirus Type 5 comprising obtaining a single-phase multi-component antiviral composition comprising from about 0.0005% to about 5.0% of at least one of sodium chlorite or potassium chlorite, based on a total weight of the single-phase multi-component antiviral composition, a buffering system, wherein pH of the single-phase multi-component antiviral composition is between 6.0 and 8.0, and water, and contacting the single-phase multi-component antiviral composition with the said virus in suspension for a contact time of at least 30 seconds, thereby reducing the initial viral load of the said virus by at least 97.3%.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the following detailed description and claims in connection with the following drawings. While the drawings illustrate various embodiments employing the principles described herein, the drawings do not limit the scope of the claims.

FIG. 1 illustrates reduction of viral load of COVID-19 virus by Oral Rinse-I in 30 seconds in accordance with various embodiments; and

FIG. 2 illustrates reduction of viral load of SARS virus by Oral Rinse-I in 60 seconds in accordance with various embodiments.

FIG. 3 illustrates reduction of viral load of Influenza A virus by Oral Rinse-I in 30 seconds in accordance with various embodiments.

FIG. 4 illustrates viral load reduction of different viruses by Oral Rinse-I in 30 seconds in accordance with various embodiments.

### DETAILED DESCRIPTION

### Definitions

The following is a list of definitions for terms used herein. Unless defined otherwise, all technical and scientific terms used herein generally have the same meaning as commonly understood by one of ordinary skill in the art. In the event that there is a plurality of definitions for a term herein, those in this section prevail unless stated otherwise. Generally, the nomenclature used herein and the laboratory procedures in cytopathicity analysis, microbial analysis, organic and inorganic chemistry, and dental clinical research are those well-known and commonly employed in the art.

The term “about” will be understood by persons of ordinary skill in the art and will vary to some extent on the context in which it can be used. Generally, “about” encompasses a range of values that are plus/minus 10% of a reference value, unless specifically defined. For instance, “about 25%” encompasses values from 22.5% to 27.5%.

As used herein, the term “acid source” means a biological material, usually a particulate material, which is itself acidic or produces an acidic environment when in contact with liquid water or solid oxychlorine anion.

As used herein, the term “ambient conditions” means approximately room temperature (e.g., 20-35° C.) and relative humidity of approximately <70%.

As used herein, the term “a reasonable period of time” means the time, ranging from months to years, depending upon the application, a composition may be expected to maintain a safe and efficacious amount of its combined ingredients without significant degradation of oxidative compound and other ingredients such as a flavoring agent of flavoring system as stated in the stability.

As used herein, “shelf-life stable” and “shelf-life stability” are used interchangeably and refer to the multi-component composition being deemed consumer acceptable after a defined period of time under ambient conditions after its production.

As used herein, “bioavailability” means the accessibility of the active agent(s) of the composition into the organic matter to which it is exposed and/or the absorption rate proportion of the dose of the composition that reaches the systemic circulation of the organic matter for which its use is intended. For example, when a composition is administered intravenously, its bioavailability is nearly 100%, while when the composition is administered topically, a fraction of the total composition reaches systemic circulation. Some embodiments described herein provide enhanced penetration or absorption of oxidative compounds when applied topically to organic matter.

As used herein, “oxidative compound” means a compound exhibiting oxidation reaction of biomolecules such as organic acids, amino acids, sulfur compounds, precursors of sulfur compounds, amino acid side chains in a protein, proteins, lipids, ribonucleic acid, deoxyribonucleic acid.

As used herein, examples of “oxidative compound” include chlorite salt, chlorite ion source, or stabilized source of chlorine dioxide.

As used herein, “biocidal”, means the property of inactivating or killing pathogens, such as viruses, bacteria, algae, yeast, fungi, archaea, and protists. Compositions that are biocidal have the property of killing a range of biological species such as bacteria, fungi, algae, yeasts, archaea, and protists and thus are not limited to one type of microbial pathogen. As used herein, “biocidal” means the effect of a composition as a treatment for reduction of viral or bacterial or fungal or microbial growth or overgrowth in fluids or biofilm which may be associated with alleviating a diseased condition or state.

As used herein, “virucidal” means the property of inactivating or killing viruses thereby either reducing or eliminating viral species.

As used herein, “fungicidal” means the property of inactivating or killing fungi.

As used herein, “bactericidal” means the property of inactivating or killing bacteria.

As used herein, “biostatic”, means the property of arresting the growth of pathogens, such as viruses, bacteria, algae, yeast or fungi, as applicable. As used herein, “biostatic” also means the property of maintaining the polymicrobial mixture of a fluid or a biofilm, as in maintaining the oral ecology so that one or more organisms have not overgrown to enable infection and disease. Compositions with biostatic attributes are useful in health maintenance, wellness and prevention of infection and disease.

As used herein, “virustatic” term means the property of inhibiting or stopping propagation of viral infection.

As used herein, “bacteriostatic” means the property of inhibiting or stopping the propagation of bacteria.

As used herein, “fungistatic” means the property of inhibiting or stopping the propagation of fungi.

As used herein, “antiviral” means the property of reducing or eliminating the number of viral count or viral load.

As used herein, “antiviral agent” means an agent that kills a virus or that suppresses its ability to infect host cells and, hence, inhibits its capability to multiply and reproduce.

As used herein, “viral load” means the quantity as measured by viral particles of a virus in a suspension of biological fluids such as blood, saliva or in the body cavity, such as found in circulatory system, the oral, nasal and urogenital cavities.

As used herein “reduction of viral load of a virus” refers to the difference between initial viral load and residual viral load after treatment with a composition. Reduction viral load of a virus is also referred as “reducing viral count of a virus”. Further, the term “reduction in initial viral load of a virus” as described herein is also commonly referred to as elimination of-, reducing infectivity of-, reducing viral count of-, destruction of- or killing of a virus in scientific and health-related literature.

As used herein, “stabilized source of chlorine dioxide,” means an aqueous solution comprised of sodium chlorite, potassium chlorite or another chlorite ion source and a compound or compounds intended to inhibit or slow the degradation of the chlorite, with resulting solution capable of releasing chlorine dioxide on its administration in the human or mammal body.

As used herein, “stabilized chlorine dioxide” is a term that is interchangeable with a stabilized source of chlorine dioxide. An example of a solution with a stabilized source of chlorine dioxide would be an aqueous solution comprised of sodium chlorite and a buffering system as defined herein.

As used herein, a “biofilm” means a biological aggregate that forms a layer on a surface of soft or hard tissues of the oral cavity, nasal passages, or ocular cavity. Biofilms comprise an aggregate comprising a community of microorganisms embedded in an extracellular matrix of polymers and/or other biomolecules such as glycoproteins. Typically, a biofilm comprises a diverse ecological community of microorganisms, including bacteria (aerobic and anaerobic), algae, protozoa, yeast, and fungi. Monospecies biofilms may also exist outside the oral and nasal cavities but do not fully represent the ecology of oral or nasal biofilms. Viruses may also be entrapped in such biofilms.

As used herein, “buffering system” means a system containing two or more agents characterized as an acid and its conjugate base or vice versa. Suitable components of buffering system may include carbonates, borates, phosphates, imidazole, citrates, acetates and mixtures thereof, and further may include any of monosodium phosphate, disodium phosphate, trisodium phosphate, alkali metal carbonate salts, imidazole, pyrophosphate salts, acetic acid, sodium acetate, citric acid, and sodium citrate. Exemplary compounds used in generating buffering systems are described in more detail in Kirk & Othmer, Encyclopedia of Chemical Technology, Fourth Edition, Volume 18, Wiley-Interscience Publishers (1996). In various embodiments, a buffering system may be used to adjust and maintain the pH of the multi-component compositions as well as to contribute to the stability of the composition for a reasonable period of time. In some embodiments, a buffering system provides a pH of about 6.0 to about 8.0 within compositions and methods described herein. Prior art referenced herein may use the term “buffer” or a “buffering agent” as a single compound intended to maintain the pH of a composition or to slow or retard the degradation of a composition. However, as used herein, such a composition with one compound or agent does not comprise a “buffering system”.

As used herein, “pH modifying agent” means an agent capable of modifying the pH of a composition. pH modifying agents comprise acidifying agents to lower pH, basifying agents to raise pH. Use of one pH modifying agent does not constitute a buffering system or buffer.

As used herein “a carrier” means those components of a composition that are capable of being commingled to provide required physical consistency and consumer goodness properties without interaction with other ingredients. Pharmaceutically-acceptable carriers may include one or more compatible solid or liquid materials, including diluents or encapsulating substances, which are suitable for topical administration to the human or animal body and provide physical action or consumer-goodness characteristics acceptable to the user.

As used herein, orally acceptable carrier means a suitable vehicle or ingredient, which can be used to apply the present compositions to the oral cavity in a safe and effective manner and that contribute to consumer goodness qualities, as defined herein.

As used herein, the term “compatible” means that the components of the composition are capable of being commingled without interaction in any manner which would significantly reduce the stability of the chlorite salt, chlorite ion source, or a stabilized source of chlorine dioxide, ingredients required for the efficacy, the carrier and excipients, and the consumer qualities of the composition.

As used herein, the terms “consumer goodness qualities” include, but are not limited to, appearance, viscosity, taste, odor, abrasiveness, color, flavor, and moisturizing attributes of the compositions deemed desirable by consumers through consumer product testing or other such means.

As used herein, a “single-phase composition” means a composition wherein all ingredients are composed in a single container at the time of composing and are not mixed with other ingredients subsequently. Thus, single-phase compositions are ready for use at any time during their shelf-life without further preparation or mixing. The bioavailability and shelf-life stability of ingredients of single-phase compositions may be determined at any point during their useful shelf-life.

As used herein, the term “dual phase composition” means a composition wherein certain ingredients are contained in one part and other ingredients are contained separately in a second part at the time of manufacture and wherein these parts are stored or packaged separately prior to use to prevent the reactivity of the chlorite salt, chlorite ion source, or a stabilized source of chlorine dioxide to the carrier and other excipients of the composition. The bioavailability of dual phase compositions may be determined once the two phases are mixed at the time of use. A fundamental difference between single-phase and dual phase compositions is how their shelf life is determined. Because the two phases of a dual phase compositions are combined just prior to usage, the shelf-life stability of dual phase compositions is the short period from the time of mixing just prior to use to the time of use. Dual-phase compositions do not have the required attribute of maintaining stability of components from the time of manufacture to the time of usage, defined as “a reasonable period of time” herein precisely because the phases of the composition are not mixed until just prior to usage. Consequently, the shelf-life stability and bio-availability of dual phase compositions comprising oxidative compounds, flavoring agents and flavoring systems, and sweetening agents are not directly comparable to single phase compositions.

As used herein, the term “essentially free” means a composition which is comprised of very low levels, below detection levels of commonly used analytical methods, of a specific ingredient, compound or molecule.

As used herein, “oral rinse” means liquid formulations, unless otherwise specified, that are used to clean the surfaces of the oral cavity. “Mouthwash,” as used in supporting literature is synonymous to oral rinse. Oral rinses may be used to promote oral hygiene, remove dental plaque and debris from the oral cavity, reduce halitosis and deliver active ingredients to help prevent dental caries, periodontal disease and systemic diseases.

As used herein, “oral spray” means liquid formulations, unless otherwise specified, that are used to clean the surfaces of the oral cavity. Oral sprays may be used to promote oral hygiene, remove dental plaque and debris from the oral cavity, reduce halitosis and deliver active ingredients to help prevent dental caries, periodontal disease and systemic diseases.

As used herein, “toothpaste” means a paste or gel dentifrice used with a toothbrush that is used to clean and maintain the aesthetics of teeth and adjacent soft tissues of the oral cavity. Toothpastes may be used to promote oral hygiene, remove dental plaque and food from teeth, reduce halitosis and deliver active ingredients to help prevent dental caries, periodontal disease and systemic diseases.

As used herein, “nasal spray” means liquid formulations, unless otherwise specified, that are used in an aerosol form to clean the nasal channel. Nasal sprays are often used to treat symptoms of sinus infection, allergies, cold and flu, and also are used as a suitable carrier for antiviral compositions and agents.

As used herein, “nasal rinse” means liquid formulations, unless otherwise specified, that are used to bath and cleanse the nasal channel. Nasal rinses may be used to relieve nasal symptoms of sinus infections, allergies, cold and flu and to wash away mucus, debris and allergens and also are used as a suitable carrier for antiviral compositions and agents.

As used herein, the term “efficacious amount” means any amount of the agent that may result in a desired biocidal or biostatic effect, a desired cosmetic effect, and/or a desired therapeutic biological effect.

As used herein, the terms “irritating” and “irritation” refer to the property of causing a local inflammatory response, such as reddening, swelling, itching, burning, or blistering, by immediate, prolonged, or repeated contact. For example, inflammation of a non-oral mucosal or dermal tissue in a mammal can be an indication of irritation to that tissue. A composition may be deemed “substantially non-irritating” or “not substantially irritating,” if the composition is judged to be slightly or not irritating using any standard method for assessing dermal, mucosal, or nasal irritation.

As used herein, the terms “pharmaceutically acceptable” as used herein is a broad term, and is to be given its ordinary and customary meaning to a person of ordinary skill in the art and refers without limitation to those compounds, materials, compositions and/or dosage forms which are, within the scope of sound medical judgment suitable for contact with the tissues of and/or for consumption by human beings and animals without excessive toxicity, irritation, allergic response, or other problem complications commensurate with a reasonable risk/benefit ratio.

As used herein, the abbreviation “ppm” means parts per million by weight or volume as applicable.

As used herein, “overgrowth” refers to excessive concentrations of viruses, bacteria, algae, yeast, and fungi leading to infection, pathogenesis and disease. Overgrowth may occur within a biofilm with reference to the other microbial members of the polymicrobial mixture. Overgrowth may refer to the oral, nasal and ocular cavities, wherein overgrowth occurs relative to the microbial mixture of the cavity.

As used herein, the term “prophylactic” means treatment administered to a subject who does not exhibit signs of a disease or exhibits early signs of the disease for the purpose of decreasing the risk of developing pathology associated with the disease.

As used herein, the term “range” means the area of variation between upper and lower limits on a particular scale. It is understood that any and all whole or partial integers between any ranges set forth herein are included herein.

As used herein, the term “safe and effective amount” means an amount of an ingredient, such as the amount of a stabilized source of chlorine dioxide, in composition of sufficient dosage to positively modify the condition to be treated, but low enough to be safe for humans and animals to use without serious side effects (at a reasonable benefit/risk ratio), within the scope of sound medical/dental judgment. “Safe and effective” pertains not only to the dosage amount but also the dosage rate (rate of release) of the chlorine dioxide applied in treatment. The safe and effective amount of a stabilized source of chlorine dioxide in a composition may vary with the particular condition being treated, the age and physical condition of the patient being treated, the severity of the condition, the duration of treatment, the nature of concurrent therapy, the specific form (e.g., salt) of the stabilized source of chlorine dioxide employed, and the particular vehicle from which the stabilized source of chlorine dioxide is applied.

As used herein, the term “stability,” means the prevention of a reaction or degradation of components, such as of a stabilized source of chlorine dioxide, comprised in a single-phase composition. A single-phase composition is “stable” if the stabilized source of chlorine dioxide of the single-phase composition is not reactive with other ingredients of the composition for a reasonable period of time as defined herein. For example, a single-phase composition is stable if it maintains consumer qualities and exhibits less than 35% loss of the chlorite salt, chlorite ion source, or stabilized source of chlorine dioxide for a period of 24 months at about 25° C. (ambient temperature) or 6 months at an accelerated temperature of 40°+2° C. and 75%±5% Relative Humidity (RH).

As used herein, “shelf life” means the length of time compositions maintain the stability of the chlorite salt, chlorite ion source, or a stabilized source of chlorine dioxide and the consumer qualities of the composition. For example, a target or stable shelf life for a composition may not comprise more than 35% loss in the concentration of chlorite salt, chlorite ion source, or stabilized source of chlorine dioxide in 6 months at 40°+2° C. and 75%±5% RH, which is equivalent to 2 years of shelf life at room temperature.

As used herein, the term “therapeutic” means intended to be administered to a subject who exhibits signs of pathology for the purpose of diminishing or eliminating those signs.

As used herein, the term “topical composition” means a product which is not intentionally swallowed or otherwise applied without recovery for purposes of systemic administration of therapeutic agents, but is retained in the nasal, anal, aural, oral, ocular, or urogenital cavities or upon the skin or other outer surfaces of the body, or upon an area of affected soft tissue for a time sufficient to contact substantially all of the surfaces and/or tissues for purposes of administration and delivery of therapeutic agents.

As used herein, the term “dispersing agent” means a compound that improves the separation of particles and prevents settling or clumping of an ingredient(s) in a multicomponent composition.

As used herein, the term “emollient agent” means a compound that reduces the loss of water from a composition.

As used herein, the term “suspending or emulsifying agent” means a compound that achieves uniform dispersion of an ingredient(s) in a single-phase composition.

As used herein, the term “fragrance” means a compound that provides a scent similar to perfume to a composition.

As used herein, the term “cooling agent” means a compound that provides a cooling, soothing, or pleasant feeling when a composition is administered in the oral cavity or nasal channel.

As used herein, the term “warming agent” means a compound that provide an olfactory sensation, especially warm sensation. Warming agents are often desired in various cosmetic preparations, such as shaving creams, hand lotions, body lotions, facial preparations, including masks, depilatories.

As used herein, the term “humectant” means a compound that preserves moisture in a composition.

As used herein, the term “thickener” means a compound that increases viscosity of a composition.

As used herein, the term “excipient” means a compound that provides physical and consumer goodness properties to a composition for its acceptance. Examples of such properties (but not limited to) are viscosity, appearance, flavor, color, thickness, sweetness, gel like structure, preservative, uniform suspension or combinations thereof.

As used herein, the term “desensitizing agent” means a compound that helps reduce or alleviate sensitivity and pain. For example, a desensitizing agent in a gel, spray, rinse or mouthwash may occlude dentin tubules or may desensitize nerve fibers, blocking the neural transmission.

As used herein, the term “surfactant” means a compound that interact with protein and lipid molecules thereby altering their spreading and wetting properties. Surfactants are compounds that reduce the surface tension between two liquids, a liquid and a gas, or a liquid and a solid. Surfactants act as detergents, wetting agents, emulsifiers, foaming agents or dispersants. Surfactants are also referred as “surface active agents”.

As used herein the term “phase stability” means a composition visually (i.e., to the unaided eye) having no liquid separation from the composition's body over a defined period of time under ambient conditions.

All percentages and ratios used herein are by weight of a single-phase composition and not of the overall topical formulation that is delivered, unless otherwise specified. All measurements are made at 25° C., unless otherwise specified. The concentration of a dissolved oxidative compound may depend on the temperatures and the range of humidity to which the solution is likely to be subjected. Heat and humidity, under normal circumstances, may cause such a composition to degrade from liquid to gas, changing its weight and rendering common assay calculations inaccurate.

### DETAILED DESCRIPTION OF THE EMBODIMENTS

The detailed description shows embodiments and uses of the compositions and methods of the present invention by way of illustration, including the best mode. While these embodiments are described in sufficient detail to enable those skilled in the art to practice the principles of the present disclosure, it should be understood that other embodiments may be realized and that logical, mechanical, chemical, and/or electrical changes may be made without departing from the spirit and scope of principles of the present disclosure. Thus, the detailed description herein is presented for purposes of illustration only and not of limitation. For example, the steps recited in any of the method descriptions may be executed in any suitable order and are not limited to the order presented.

With regard to procedures, methods, techniques, and workflows that are in accordance with some embodiments, some operations in the procedures, methods, techniques, and workflows disclosed herein may be combined and/or the order of some operations may be changed.

In various embodiments, a single-phase, multi-component composition comprises an oxidative compound such as, for example, at least one of, ammonium peroxydisulfate, carbamide (urea) peroxide, ferric chloride, hydrogen peroxide, potassium bromate, potassium chlorate, potassium perchlorate, potassium dichromate, potassium ferricyanide, potassium peroxymonosulfate, potassium persulfate, sodium bromate, sodium chlorate, sodium perchlorate, sodium chlorite, sodium hypochlorite, sodium iodate, sodium perborate, sodium percarbonate, sodium persulfate, stabilized chlorine dioxide, strontium peroxide, and zinc peroxide.

In some aspects, a single-phase composition multi-component comprises an oxidative compound comprising a chlorite salt (e.g., sodium chlorite, potassium chlorite, or combinations thereof) or a stabilized source of chlorine dioxide. In some embodiments described herein, the aforementioned compound for a single-phase multi-component composition may be selected from at least one of a low-molecular-weight compound, a compound of suitable size and properties to react with macromolecules like proteins, amino acids, lipids, nucleic acids, carbohydrates on the virus or cell surface of microorganisms and to permit diffusion or uptake through surface layer of virus or cell wall of microorganism to react with internal components and a compound which stimulates apoptotic or necrotic cell death.

In some aspects, a single-phase composition is comprised of chlorite salt (e.g., sodium chlorite, potassium chlorite, or combinations thereof) or a stabilized source of chlorine dioxide. In some embodiments described herein, the aforementioned compound for a single-phase multi-component composition may be selected from at least one of a low-molecular-weight compound, a compound of suitable size and properties to react with macromolecules like proteins, amino acids, lipids, nucleic acids, carbohydrates on the virus or cell surface of microorganisms and to permit diffusion or uptake through surface layer of virus or cell wall of microorganism to react with internal components and a compound which stimulates apoptotic or necrotic cell death.

In some embodiments, the oxidizing threshold is low, indicating that the selected chlorite salt, chlorite ion source, or stabilized source of chlorine dioxide interact strongly with its target by chemical rather than physical means.

In some embodiments, the single-phase, multi-component composition reduces viral load of viruses infecting humans, animals and birds. In some embodiments, the single-phase, multi-component composition reduces viral load of one or more pathogenic viruses or virus types, for example, coronaviruses, human coronaviruses, Influenza virus, HCoV-229E, HCoV-NL63, HCoV-0C43, HCoV-HKU1, SARS-CoV, MERS-CoV, SARS-CoV-2, and Influenza A. In some embodiments, the single-phase, multi-component composition comprises a rinse, solution, spray, paste or gel. In some embodiments, the single-phase, multi-component composition does not exhibit cytotoxicity to the host cells.

In some embodiments, a single-phase multi-component composition is comprised of about 0.005% to about 0.8% of a chlorite salt, a chlorite ion source or a stabilized source of chlorine dioxide. In another embodiment, the single-phase multi-component composition is comprised of about 0.05% to about 0.5% aforementioned oxidative compound. In another embodiment, the single-phase multi-component composition is comprised of about 0.005% to about 2.0% aforementioned oxidative compound.

In some aspects, the single-phase composition may further comprise a carrier. In some embodiments, the selected carrier may not substantially reduce either the stability of the composition or its efficacy. In further embodiments, the selection of suitable carrier(s) may depend on considerations such as compatibility with the ingredients required for the efficacy, consumer goodness qualities, cost, and contribution to shelf-life stability. Examples of carriers include gelling agents, whitening agents, flavoring agents and flavoring systems, coloring agents, abrasive agents, foaming agents, desensitizing agents, dispersants, humectants, sweetening agents analgesic and anesthetic agents, anti-inflammatory agents, anti-malodor agents, anti-microbial agents, anti-plaque agents, anti-viral agents, biofilm disrupting, dissipating or inhibiting agents, cellular redox modifiers, antioxidants, cytokine receptor antagonists, dental anti-calculus agents, fluoride ion sources, hormones, metalloproteinase inhibitors, enzymes, immune-stimulatory agents, lipopolysaccharide complexing agents, tissue growth factors, vitamins and minerals, water, and mixtures thereof.

In some aspects, the single-phase multi-component composition is comprised of a buffering system. The buffering system is required to achieve and maintain a pH of the single-phase composition in the range required to prevent the degradation of chlorite salt, chlorite ion source, or a stabilized source of chlorine dioxide in the single-phase composition. A buffering system may also be useful to adjust the pH to the desired level to achieve consumer goodness properties and to maintain a pH of about 6.0 to about 8.0. A buffering system differs from a single pH modifying agent used to reduce the pH of a composition, in that, while it may be used to raise or lower pH to a desired level during comprising the composition, it is also useful to maintaining the shelf-life stability and bioavailability of ingredients for a reasonable period of time, as defined herein.

In some embodiments, a buffering system in the single-phase multi-component composition is comprised of about 0.01% to about 6.0% of a base compound and from about 0.001% to about 4.0% of an acidic compound.

In some aspects, the single-phase multi-component composition is comprised of one or more pH modifying agents. pH modifying agents among those useful herein include acidifying agents to lower pH, basifying agents to raise pH and buffering agents to control pH within a desired range. For example, one or more compounds selected from acidifying, basifying and buffering agents can be included to provide a pH of 2 to 10, or in various embodiments from about 2 to about 8, from about 3 to about 9, from about 4 to about 8, from about 5 to about 7, from about 6 to about 10, from about 6 to about 8, from about 7 to about 8, and from about 7 to about 9. Any orally or nasal channel acceptable pH modifying agent is comprised of carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, disodium phosphate, trisodium phosphate, pyrophosphate salts, etc.), imidazole and mixtures thereof. One or more pH modifying agents are optionally present in a total amount effective to maintain the composition in an orally acceptable pH range. In some embodiments, the single-phase composition may include from about 0.01% to about 10% pH modifier agents based on a total weight of the oral care composition.

In some aspects, the single-phase multi-component composition is comprised of an additional active ingredient. In some embodiments, an additional active ingredient is comprised of a least of one of, a fluoride ion source, anti-microbial agent, analgesic compound, anti-inflammatory agents, anti-malodor agents, anti-plaque agents, anti-viral agents, biofilm disrupting, dissipation or inhibiting agents, hormones, enzymes, metalloproteinase inhibitors, immune-stimulatory agents, lipo and a numbing agent. In further embodiments, the multi-component composition is comprised of at least one of, an excipient including any of water, abrasives, humectants, thickeners, sweeteners, moisturizers, flavors, colors, fillers, and extenders.

In some aspects, the single-phase multi-component composition is comprised of a pharmaceutically acceptable carrier and/or excipients. The pharmaceutical carriers and/or excipients selected are comingled with chlorite salt, chlorite ion source, or a stabilized source of chlorine dioxide in a single-phase composition without interaction in any manner that would reduce the stability of the said compound, the flavoring system, the consumer goodness qualities, the safety and effectiveness of the composition in treating or preventing anal, aural, oral, nasal, ocular, urogenital, foot, and skin disorders, or diseases of the skin or foot and the inflammation and infection of tissues therein. The choice of a pharmaceutically acceptable carrier and/or excipient may be determined by the way the composition is to be introduced into the oral, nasal, or aural cavity. The selection of a pharmaceutically acceptable carrier and/or excipient may depend on secondary considerations such as, but not limited to, consumer goodness qualities, the flavoring system, the buffering system, costs and shelf-life stability.

In embodiments, the pharmaceutically acceptable carrier and/or excipients is comprised of an amount of from about 0.01% to about 30%, for example, from about 0.01%, 0.1%, 1%, 2%, 3%, 4%, 5%, 6%, 7%, 8%, 9% or to about 10% by weight or volume of the single-phase composition. In other embodiments, the pharmaceutically acceptable carrier is comprised of an amount from about 0.01% to about 60%, from about 0.01% to about 30%, or from about 0.01% to about 20%.

In aspects, the single-phase multi-component composition is comprised of an alkali metal bicarbonate salt. Alkali metal bicarbonate salts are soluble in water and, unless stabilized, tend to degrade oxidative compounds in an aqueous system. Sodium bicarbonate, also known as baking soda, may be comprised as an alkali metal bicarbonate salt into the single-phase composition. In embodiments, the alkali metal bicarbonate salt is comprised of an amount of from about 0.01% to about 70%, for example, from about 0.01%, 0.1%, 1%, 5%, 10%, 20%, 30%, 40%, 50%, 60%, or to about 70% by weight of the single-phase composition. In some other embodiments, the alkali metal bicarbonate salt is comprised of an amount from about 0.5% to about 70%, from about 1% to about 50%, or from about 5% to about 50%.

In embodiments, the anti-calculus agent is comprised of an amount of from about 0.01% to about 50%, for example, from about 0.01%, 0.1%, 1%, 5%, 10%, 15%, 20%, 25%, 30%, 40%, or to about 50% by weight of the single-phase composition. In other embodiments, the anti-calculus agent is comprised of an amount from about 0.5% to about 25%, from about 1% to about 25%, or from about 5% to about 50%.

In some aspects, a single-phase multi-component composition is comprised of a coloring agent. The consumer goodness quality of coloring may not be degraded by the chlorite salt, chlorite ion source, or a stabilized source of chlorine dioxide and vice versa. Coloring enables the consumer to more readily ascertain usage and dosage. Certain colors of the composition may be deemed undesirable for certain anal, aural, ocular, oral or urogenital applications. In some embodiments, a coloring agent may include, FD&C Blue No. 1 or titanium dioxide. Suitable coloring agents are those that are stable and do not degrade in the presence of the aforementioned compounds within the compositions and do not degrade the aforementioned compounds. In some embodiments, the coloring agent is comprised of an amount of from about 0.01% to about 10.0% by weight of the single-phase composition.

In some aspects, a single-phase multi-component composition is comprised of a cooling and/or warming agent. Suitable cooling and/or warming agents may be those that are stable and do not degrade the presence of chlorite salt, chlorite ion source, or a stabilized source of chlorine dioxide within the compositions.

In some aspects, the single-phase multi-component composition is comprised of a flavoring agent and/or flavoring systems. Suitable flavoring agents are those that are stable and do not degrade in the presence of the chlorite salt, chlorite ion source, or a stabilized source of chlorine dioxide within the compositions and do not degrade aforementioned compounds. The flavoring systems may be emulsified to generate a flavoring system for protecting the flavoring agent from degradation by its reaction with chlorite salt, chlorite ion source, or stabilized source of chlorine dioxide. The flavoring system as taught by E.P. Patent Publication No. 2,654,902 may be used in various embodiments. In some embodiments, a flavoring agent may be selected from menthol, mint oil, emulsified mint oil, bubblegum flavor, or berry flavor. In some embodiments, the flavoring agent may be in an amount of from about 0.01% to about 10% by weight of the single-phase composition.

In some aspects, the single-phase multi-component composition is comprised of a sweetening agent. The sweetening agent may be stable and not degrade in the presence of the oxidative compound or degrade chlorite salt, chlorite ion source, or a stabilized source of chlorine dioxide. In some embodiments, the sweetening agents are comprised of, but not limited to, sucrose, aspartame, acesulfame, stevia, saccharin; saccharin salts, especially sodium saccharin; sucralose, sodium cyclamate, and mixtures thereof. In some embodiments, sweetening agents that are polyhydroxy alcohols such as xylitol, mannitol, and sorbitol may not be comprised in the single-phase composition since such compounds may react with chlorite salt, chlorite ion source, or stabilized source of chlorine dioxide making the composition unstable. In various embodiments, a single-phase composition is free of polyhydroxy sweeteners such as xylitol, mannitol, and sorbitol. In some embodiments, a sweetening agent is comprised of sucrose, sucralose, acesulfame, aspartame, cyclamate, or saccharin. In some embodiments, the sweetener is comprised of an amount of from about 0.01% to about 0.5% by weight of the single-phase composition.

In some aspects, a single-phase multi-component composition further is comprised of one or more humectants. A humectant serves to keep gels and suspensions from hardening or losing their consumer goodness qualities when exposed to air, to add to the compositions a moist feel to the consumer goodness qualities and, for particular humectants orally applied, to impart a desirable sweetness of flavor, such as gel compositions. In some embodiments, the humectant in a single-phase composition may not include polyhydroxy compounds such as polyhydroxy alcohols, including arabitol, erythritol, glycerol, maltitol, mannitol, sorbitol, and/or xylitol. Other compounds which provide moist texture for suitable formulations, as described herein, may also be used.

In some embodiments, the humectant is comprised in an amount of about 0.001% to about 70%, for example, from about 0.001%, 0.01%, 1%, 2%, 5%, 10%, 20%, 30%, 40%, 50%, or to about 70% by weight or volume of the single-phase composition. In some other embodiments, the humectant may be in an amount from about 1% to about 15%, from about 15% to about 55%, or from about 25% to about 55%.

In some aspects, the single-phase multi-component composition is comprised of a fluoride ion source. In some embodiments, the single-phase composition may include free fluoride ions or covalently bound fluorine in a form that may be hydrolyzed by oral enzymes to yield free fluoride ions. Free fluoride ions may be provided, for example, by sodium fluoride, silver diamine fluoride, stannous fluoride, or indium fluoride. Covalently bound fluorine, which can be enzymatically hydrolyzed to yield free fluoride, may be provided by sodium monofluorophosphate. In various embodiments, sodium fluoride may be comprised in the single-phase composition as the source of free fluoride ions. If a fluoride ion source is used as a component in a single-phase composition, a “fluoride ion source” may be preferred.

In some embodiments, a single-phase multi-component composition further is comprised of a source of fluoride ion that yields fluoride ions from about 0 ppm to about 5000 ppm, or from about 50 ppm to about 3500 ppm, from about 500 ppm to about 3500 ppm. In some embodiments, the fluoride ion source is comprised of an amount of from about 0% to about 2.0%, for example, from about 0.01%, 0.1%, 0.15%, 0.2%, 0.3%, 0.4%, 0.5%, 0.6%, 0.7%, 0.8%, or to about 2.0% by weight or volume of the single-phase composition. In other embodiments, the fluoride ion source is comprised of an amount from about 0.0% to about 0.03%, from about 0.0% to about 0.7%, from about 0.1% to about 0.8%, from about 0.01% to about 0.07%, or from about 0.0% to about 0.8%.

In some aspects, a single-phase multi-component composition is comprised of a thickening or binding agent. The thickening or binding agent provide desired consumer goodness qualities appropriate to the carrier, such as the desirable consistency or viscosity of the composition, to provide desirable dosage and rate of release of the oxidative compounds upon use, and to adhere to hard or soft tissues in a topical application. Examples of thickening or binding agents are carboxyvinyl polymers, seaweed derivatives such as carrageenan, hydroxyethyl cellulose, laponite, powdered polyethylene, or water-soluble salts of cellulose ethers such as sodium carboxymethylcellulose and sodium carboxymethyl hydroxyethyl cellulose. Natural gums such as gum karaya, guar gum, xanthan gum, gum arabic, and gum tragacanth can also be used. Colloidal magnesium aluminum silicate or finely divided silica may be used as part of the thickening or binding agent to further improve texture. Higher concentrations of thickening agents can be used for chewing gums, lozenges (including breath mints), sachets, non-abrasive gels and gels intended for use in wound-healing, urogenital or oral disease.

In some embodiments, the thickening or binding agent is comprised of an amount of from about 0% to about 15%, for example, from about 0.01%, 0.1%, 1%, 2%, 3%, 4%, 5%, 6%, 7%, 8%, or to about 15% by weight or volume of the single-phase composition. In some other embodiments, the thickening or binding agent is comprised of an amount from about 0.1% to about 15%, from about 2.0% to about 10%, from about 4% to about 8%, from about 1.0% to about 4.0%, or from about 5.0% to about 7.0%.

In aspects, the single-phase multi-component composition further is comprised of water. Water may provide the remaining weight percent of the single-phase compositions (i.e., the weight percent not attributed to the other components described herein). Water used in the single-phase compositions used as commercially suitable topical compositions can be of low ion content and essentially free of organic impurities. Water is comprised of up to about 98% of the composition, particularly for mouthwashes, mouth rinses and mouthwashes, oral and nasal sprays, vaginal douches, and soaks, and preferably from about 5% to about 60%, by weight of the aqueous compositions herein. These amounts of water comprise the free water which is added to the composition plus that which is introduced with other materials comprising the composition. Some embodiments of single-phase compositions described herein, such as powders, lozenges and chewing gum, are of course essentially free of or contain only small amounts of water.

In aspects, the single-phase multi-component composition further is comprised of a surfactant. Surfactants may be anionic, cationic, non-ionic, or amphoteric (zwitterionic). These may be useful as foaming agents in oral care, cosmetic, healthcare, and pharmaceutical products. Such foaming agents may also useful in the retention of sanitizing and moisturizing agents in skin care products, such as shaving creams and foams. In certain embodiments, the surfactant is comprised of an amount of from about 0% to about 15%, for example, from about 0.01%, 0.1%, 1%, 2%, 3%, 4%, 5%, 6%, 7%, 8%, or to about 15% by weight or volume of the composition. In some other embodiments, the surfactant is comprised of an amount from about 0.1% to about 15%, from about 2.0% to about 10%, or from about 4% to about 8%.

In aspects, the single-phase multi-component composition further is comprised of a desensitizing agent. The desensitizing agent may be provided for temporary relief from pain to hard or soft tissues. In certain embodiments, the desensitizing agent is comprised of compounds such as strontium chloride, strontium acetate, arginine, hydroxyapatite, nano-hydroxyapatite (nano-HAp), calcium sodium phosphosilicate, potassium chloride or potassium nitrate. In various embodiments, the compositions are essentially free of compounds that may irritate sensitive body cavities such as anal, nasal, ocular, oral, and urogenital, such as sodium lauryl sulfate. Examples of sensitivities and resultant diseases of the oral cavity include canker sores, oral mucositis, and dry mouth.

In aspects, the single-phase multi-component composition further comprises a preservative. In embodiments, the preservative comprises a methyl paraben, propyl paraben, disodium EDTA, benzyl alcohol, benzoic acid, sodium benzoate or potassium sorbate. In embodiments, the preservative may be present in an amount of from about 0% to about 2%, for example, 0.01%, 0.1%, 1%, or 2% by weight or volume of the composition. In other embodiments, the surfactant may be in an amount from about 0.1% to about 0.15%, from about 0.2% to about 1%, from about 0.01% to 0.5%, or from about 0.4% to about 0.8%.

The single-phase multi-component composition does not contain a polyhydroxy compound. Polyhydroxy compounds are known to react and degrade chlorite salt, chlorite ion source, or stabilized source of chlorine dioxide and therefore, may be excluded from the single-phase composition. Polyhydroxy compounds may include glycerin, alcohols, polyethylene glycols, xylitol, and sorbitol.

In some aspects, the single-phase multi-component composition is formulated as a cosmetic. Cosmetic compositions (for example, a solid cosmetic composition, such as a gel, soft-solid or semi-solid (cream), or stick), may be comprised of a base composition containing at least one silicone fluid (for example, silicone liquids such as silicone oils) which is thickened using a siloxane or silicon-based polyamide as a gelling agent; a carrier in which cosmetically active materials are incorporated; and at least one active ingredient to provide the activity for such cosmetic composition. in some embodiments, the cosmetic compositions are transparent to visible light (clear), including solid transparent (clear) compositions. In some embodiments, the cosmetic composition is formulated that the final composition is opaque. In some embodiments, the cosmetic composition is formulated that the final composition is not transparent.

In some embodiments, the cosmetic is comprised further of one or more additional cosmetic or therapeutic agents as carriers of the composition, selected from the group comprising abrasive polishing materials, alkali metal bicarbonate salts, analgesic and anesthetic agents, anti-inflammatory agents, anti-malodor agents, anti-microbial agents, anti-fungicidal agents, anti-plaque agents, and anti-viral agents, biofilm disrupting, dissipating or inhibiting agents, buffers and buffering systems, cellular redox modifiers and antioxidants, coloring agents and coloring systems, flavoring agents and flavoring systems, cytokine receptor antagonists, dental anti-calculus agents, hormones, metalloproteinase inhibitors, immune-stimulatory agents, lipopolysaccharide complexing agents, tissue growth factors, titanium dioxide, vitamins and minerals, and mixtures thereof. Cosmetic embodiments may possess therapeutic as well as cosmetic effects. It is recognized that in certain forms of therapy, such as combinations of therapeutic agents in the same delivery system, may be useful in order to obtain an optimal effect. In some embodiments, the single-phase composition may be combined with one or more such agents in a single-phase delivery system to provide combined effectiveness, while maintaining the stability of chlorite salt, chlorite ion source, or a stabilized source of chlorine dioxide.

In some embodiments, the single-phase multi-component composition may be specifically formulated for use in humans or for use in animals, for example in the form of rinses, gels, creams, washes, sprays, lozenges, therapeutic floss, tape, patches, compresses, or strips, for use in skin care, oral care, nasal care and as a solution used in irrigation devices for use in the oral and other body cavities. These embodiments may vary, for example, when formulated for humans and when formulated for horses or dogs.

In some aspects, the single-phase multi-component composition has consumer goodness qualities. In some embodiments, it is important to select ingredients for an oral care composition that achieve a desirable range of viscosity to ensure product manufacturability, stability, and quality, as well as consumer acceptance. In some embodiments, the single-phase composition may be phase stable as defined herein. In other words, a consumer quality of a liquid embodiment, such as an oral rinse, may be one where the composition retains clarity (clear, water-like appearance); however, clarity is not limited by the presence of a color in the composition if a color is intended. For another example, a nasal spray, or an oral spray embodiment should not sting, stain, burn or otherwise cause irritation to the user, has a viscosity that enables ease of use, and has a pleasing fragrance or no fragrance at all following use.

In some aspects, the single-phase multi-component composition is suitable for a variety of indications, as well as oral, ocular, and nasal and other topical uses. Suitable topical indications include oral, nasal, ocular, and skin-care conditions and diseases. The combination may be suitable in select single-phase compositions comprising antimicrobial, antiseptic, antioxidant, fungicidal and fungistatic, virucidal and virustatic, bactericidal and bacteriostatic, biofilm penetration, biofilm dissipation and reduction, coagulant, deodorant, desensitizing, disinfectant, fungicidal and fungistatic, herbicidal, tissue damage reduction, bleaching, stain removal, and tooth whitening active ingredients and excipients.

In some aspects, the single-phase multi-component composition maintains stability and consumer goodness as defined herein from manufacture of the composition through twelve (12) months storage under ambient conditions, corresponding to its intended industrial use. In some embodiments, the single-phase composition may exhibit no more than 10% loss in a stabilized source of chlorine dioxide in in three (3) months at 40°+2° C. and 75%±5% relative humidity (RH) which may be equivalent to twelve months at room temperature so as to induce delivery of its intended therapeutic indications and/or cosmetic attributes In some embodiments, the single-phase composition may exhibit no more than 20% loss in stabilized source of chlorine dioxide in three (3) months at 40°+2° C. and 75%±5% relative humidity (RH). In some embodiments, the multi-component composition may exhibit no more than 30% loss in a stabilized source of chlorine dioxide in three (3) months at 40°+2° C. and 75%±5% relative humidity (RH). In some embodiments, the multi-component composition may exhibit no more than 40% loss in a stabilized source of chlorine dioxide in three (3) months at 40°+2° C. and 75%±5% relative humidity (RH) In another embodiment, storage of the composition under accelerated conditions (typically 40°+2° C. and 75%±5% relative humidity, RH) can project real time suitability of a composition for consumer use, anticipating the time of manufacture, transit from point of manufacture to wholesaler, from wholesaler to retailer, from retailer to consumer, plus the anticipated storage time by the consumer as the product is consumed.

In one aspect of the disclosure, methods of reducing viral load are disclosed. In embodiments, the methods comprise contacting a virus with a single-phase, multi-component antiviral composition as described herein. In some embodiments, the contacting occurs for about 30 seconds, about 60 seconds or up to 120 seconds. In some embodiments, the virus is capable of infecting humans, animals and birds. In some embodiments, comprises one or more pathogenic viruses or virus types, for example, coronaviruses, human coronaviruses, Influenza virus, HCoV-229E, HCoV-NL63, HCoV-0C43, HCoV-HKU1, SARS-CoV, MERS-CoV, SARS-CoV-2, and Influenza A. In some embodiments, the method further comprises formulating the single-phase, multi-component composition as a rinse, solution, spray, paste or gel.

In embodiments, the method results in a reduction of initial viral load of SARS-CoV-2 by between about 96.3% and about 99.9%. In embodiments, the method results in a reduction of initial viral load of SARS-CoV by between about 72.4% to 91.2%. In embodiments, the method results in a reduction of initial viral load of HCoV-229E by up to 82.22%. In embodiments, the method results in a reduction of initial viral load of Influenza A by up to 99.998%.

### Various Embodiments of Compositions and Testing of Compositions

Various embodiments of the composition taught herein are presented below. It is important to understand, as discussed above, that various oxidative compounds may have some degree of antimicrobial and/or antiviral activity, but the extent of this activity may vary greatly depending upon the oxidative compound, the virus and/or microbe, and other factors. In that regard, it is difficult or impossible to extrapolate the effect of an oxidative compound on one particular type of virus based upon the oxidative compound's effect on a different virus. Indeed, as is shown here, the efficacy of various compositions disclosed herein may vary greatly depending upon the particular virus to which it is applied. In that regard, various embodiments demonstrate a surprisingly high degree of efficacy as measured by reduction in viral load achieved in a surprisingly short contact time. Stated another way, the precise degree of viral load reduction achieved by various compositions in accordance with the present disclosure is unexpected with respect to certain viruses. Testing of various embodiments as presented below demonstrates the capacity of various compositions in accordance with the present disclosure to disinfect the oral, nasal, ocular, and aural cavities of humans and animals and for the deactivation of Influenza A virus, the Middle East Respiratory Syndrome virus (MERS-CoV or MERS virus), Severe Acute Respiratory Syndrome virus (SARS-CoV or SARS virus), and Coronavirus disease-19 virus (SARS-CoV-2, COVID-19 virus or 2019-nCoV) in those cavities.

### Exemplary Composition I: Oral Rinse Embodiments

Various single-phase multi-component oral care rinse compositions are comprised of: about 0.0005% to about 5.0% of chlorite salt such as sodium chlorite or potassium chlorite, a buffering system comprising from about 0.02% to about 4.0% base, such as disodium hydrogen phosphate or trisodium phosphate, from about 0.01% to about 2.10% acid, such as sodium dihydrogen phosphate, phosphoric acid, citric acid or acetic acid, from about 0.001% to about 0.5% sweetening agent such as sucrose, acesulfame, aspartame, cyclamate, sucralose, or saccharin, from about 0.0025% to about 1.2%, a flavoring agent or a flavoring system comprising flavoring, such as menthol, mint oil, emulsified mint oil, tropical fruit, bubblegum, watermelon, strawberry or berry flavor, from about 0.001% to about 0.07%, fluoride ion source or source of releasable fluoride ion, such as sodium fluoride, stannous fluoride, sodium monofluorophosphate, or acidulated phosphate fluoride, and water to 100% thereby maintaining the final pH in the range of 6.0 to 8.0. For preparing fluoride-free oral rinse compositions, the fluoride ion source is eliminated from the composition and the quantity of water is adjusted accordingly. Similarly, for preparing fluoride-free and unflavored oral rinse compositions, the fluoride ion source, the flavoring agents, and sweeteners are eliminated from the composition.

### Exemplary Composition II: Oral Spray Embodiments

Various oral care spray formulations are comprised of: from about 0.0005% to about 2.0% chlorite salt such as sodium chlorite, from about 0.02% to about 4.0%, a buffering system comprising a base, such as disodium hydrogen phosphate, sodium citrate, or trisodium phosphate, from about 0.001% to about 0.2% and an acid or a buffering salt on the acidic side, such as phosphoric acid, citric acid, acetic acid, or sodium dihydrogen phosphate, from about 0.001% to about 0.5%, sweetening agents such as sucrose, acesulfame, aspartame, cyclamate, sucralose, or saccharin, from about 0.1% to about 7.5%, flavoring agents or a flavoring system comprising flavoring agents, such as menthol, mint oil, emulsified mint oil, watermelon, bubblegum, tropical fruit, strawberry or berry flavor, from about 0.05% to 7.0% dispersing agent such as a polysorbate, and water to 100% thereby maintaining the final pH in the range of 6.0 to 8.0. Optional ingredients in oral spray embodiments are from 0.0001% to 0.5% preservatives, such as methyl paraben, propyl paraben, disodium EDTA, sodium benzoate, benzoic acid or combination thereof.

### Exemplary Composition III: Nasal Spray Embodiments

Various nasal channel care spray formulations are comprised of: from about 0.00005% to about 1.0% chlorite salt such as sodium chlorite, from about 0.0001% to about 4.0% a base such as disodium hydrogen phosphate, sodium citrate, or trisodium phosphate, from about 0.0001% to about 0.2% an acid or a buffering salt on the acidic side, such as phosphoric acid, citric acid, acetic acid, or sodium dihydrogen phosphate, from about 0.05% to 7.0% dispersing agent such as a polysorbate, 0.05% to 5.0% salt such as sodium chloride or potassium chloride, and water to 100% thereby maintaining the final pH in the range of 6.0 to 8.0. Optional ingredients in oral spray embodiments are from 0.0001% to 0.5% preservatives, such as methyl paraben, propyl paraben, disodium EDTA, sodium benzoate, benzoic acid or combination thereof, from about 0.001% to about 0.5%, sweetening agents such as sucrose, acesulfame, aspartame, cyclamate, sucralose, or saccharin, and from about 0.05% to about 7.5%, flavoring agents or a flavoring systems comprising flavoring agents, such as menthol, mint oil, emulsified mint oil, watermelon, bubblegum, tropical fruit, strawberry or berry flavor.

In various embodiments, various single-phase nasal spray formulations are comprised of from about 0.005% to about 1.0% chlorite ion source such as sodium chlorite, from about 0.01% to about 0.5% a base such as disodium hydrogen phosphate, sodium citrate, or trisodium phosphate, from about 0.01% to about 0.05% an acid or a buffering salt on the acidic side, such as phosphoric acid, citric acid, acetic acid, or sodium dihydrogen phosphate, from about 0.01% to about 1.0% an N-acyl sarcosinate compound such as sodium lauroyl sarcosinate, sodium cocoyl sarcosinate, or sodium myristoyl sarcosinate, from about 0.1% to 5.0% dispersing agent such as a polysorbate, 0.05% to 5.0% salt such as sodium chloride or potassium chloride, from 0.01% to 0.5% preservative, such as methyl paraben, propyl paraben, disodium EDTA, sodium benzoate, potassium sorbate or combination thereof and water to 100% thereby maintaining the final pH in the range of about 6.0 to about 8.0. The compositions are comprised of a buffering system as described herein.

### Exemplary Composition IV: Oral Care Gel Embodiments

Various single-phase oral care gel compositions are comprised of: from about 0.005% to about 2.0% chlorite salt such as sodium chlorite, from about 0.7% to about 4.2%, a buffering system comprising a base, such as disodium hydrogen phosphate or trisodium phosphate, from about 0.06% to about 2.20%, and an acid such as phosphoric acid, sodium dihydrogen phosphate, citric acid, or acetic acid, from about 5.0% to about 7.0% gelling agent such as gelatin, pectin, xanthan gum, guar gum, cellulose gum, other natural or synthesized gums, or sodium carboxymethyl cellulose, from about 0.05% to about 0.5%, sweetening agents, such as sucrose, acesulfame, aspartame, sucralose, or saccharin, from about 0.025% to about 1.2%, flavoring agents or a flavoring systems comprising flavoring agents, such as menthol, mint oil, emulsified mint oil, bubblegum flavor, strawberry, multi-fruit, watermelon or berry flavor, from about 0.01% to about 0.8% fluoride ion source such as sodium fluoride, stannous fluoride, or sodium monofluorophosphate, and water to 100% thereby maintaining the final pH in the range of 6.0 to 7.8. For preparing fluoride-free gel compositions, the fluoride ion source is eliminated from the composition and the quantity of water is adjusted accordingly.

### Exemplary Composition V: Toothpaste Embodiments

Various single-phase oral care toothpaste compositions are comprised of: from about 0.005% to about 2.0% of a chlorite ion source such as sodium chlorite, from about 0.7% to about 4.2% a base, such as disodium hydrogen phosphate or trisodium phosphate, from about 0.05% to about 2.20% of an acid or a buffering salt with an acidic pH, such as sodium dihydrogen phosphate, citric acid, or acetic acid, from about 0.2% to about 5.0% of an N-acyl sarcosinate compound, such as sodium lauroyl sarcosinate, sodium cocoyl sarcosinate, or sodium myristoyl sarcosinate, from about 0.8% to about 1.1% of a coloring agent such as FD&C Blue No. 1 or titanium dioxide, from about 1.0% to about 4.0% of a gelling agent such as gelatin, pectin, guar gum, xanthan gum, other natural or synthesized gums, cellulose gum or sodium carboxymethyl cellulose, from about 20.0% to about 70.0% of an abrasive agent such as hydrated silica, calcium hydrogen phosphate, alumina, sodium bicarbonate, from about 0.05% to about 0.5% of a sweetening agent such as sucrose, sucralose, acesulfame, aspartame, cyclamate, or saccharin, from about 0.025% to about 1.2% of a flavoring agent such as menthol, mint oil, emulsified mint oil, tropical fruit, watermelon, bubblegum, strawberry or berry flavor, from about 0.0% to about 0.8% of a fluoride ion source or source of releasable fluoride ion, such as sodium fluoride, silver diamine fluoride, sodium monofluorophosphate, or stannous fluoride, and water to 100%, thereby maintaining the final pH in the range of about 6.0 to about 8.0. For preparing fluoride-free toothpaste compositions, the fluoride ion source is eliminated from the composition and the quantity of water is adjusted accordingly.

### Methods for Preparing Exemplary Compositions

In preparing a single-phase composition where the Exemplary Composition is a liquid, the base compound selected may be dissolved in deionized or purified water in a separate preparation. This solution may be mixed with the chlorite salt in an aqueous solution. The remaining ingredients, e.g., sweetening agents, flavoring agents or flavoring system, fluoride ion source, additional deionized or purified water, and/or other ingredients as described above and as applicable, may be added in appropriate amounts. The buffering system, comprising an appropriate amount of weak acid may be dissolved in water and the appropriate quantity may be mixed with the composition to maintain the final pH of the overall formulation in the range of 6.0 to 8.0. The composition may be stirred for about 45 minutes for achieving homogeneity. All compounding may be required to occur at ambient temperatures to maintain the stability of the composition.

Similarly, in preparing a multi-component composition where the Exemplary Composition is an aerosol liquid spray or an oral spray as defined herein, the method for preparation may follow the method for oral rinse composition taught above, wherein additional ingredients such as dispersing agents, humectants, or preservatives may be mixed with the composition to adjust the pH of the final composition to the range of 6.0 to 8.0.

In preparing compositions as described herein and where the Exemplary Composition is a paste or gel, the gelling agents may be dissolved in water. Pharmaceutically-acceptable buffering system comprising compounds of the appropriate type and concentration such as weak acid and its conjugate base or weak base and its conjugate acid may then be added to the solution of gelling agent in water until the preferred final pH range of 6.0 to 8.0 is achieved. Then the solution containing a buffering system may be mixed with the chlorite ion source in an aqueous solution. The remaining ingredients, e.g., humectants, sweetening agents, coloring agents, abrasive agents, fluoride ion source, flavoring agent(s), emollient agents, suspending or emulsifying agents, additional deionized or purified water, and other ingredients as described above and as applicable, may be added in appropriate amounts to maintain the final pH of the overall formulation in the range of 6.0 to 8.0. All compounding may occur at ambient temperatures to maintain the stability of the composition.

### EXAMPLE 1

### Formulation of Oral Rinse Oral Spray and Toothpaste Embodiments

Various compositions of Exemplary Compositions I, II and V were formulated as described and were tested. The ingredients of the compositions are presented in Table 1 and Table 2. Oral Rinse-I, Oral Rinse-II and Oral Rinse-III were prepared as taught in Exemplary Composition I. Oral Spray-X and Oral Spray-Y were prepared as taught in Exemplary Composition II. Toothpaste C, Toothpaste D and Toothpaste K were prepared as taught in US patent application Ser. Nos. 16/133,359 and 17/094,489.

TABLE 1
Comparison of Oral Care Compositions Ingredients

| | Oral | Oral | Oral | Oral Spray- | Toothpaste | Toothpaste | Toothpaste |
|---|---|---|---|---|---|---|---|
| Ingredient | Rinse-I | Rinse-II | Rinse-III | X and Y | C | D | K |
| Stabilized | Sodium | Sodium | Sodium | Sodium | Sodium | Sodium | Sodium |
| source of | Chlorite | Chlorite | Chlorite | Chlorite | Chlorite | Chlorite | Chlorite |
| Chlorine | | | | | | | |
| Dioxide | | | | | | | |
| (i.e., | | | | | | | |
| Chlorite | | | | | | | |
| Ion Source | | | | | | | |
| only) | | | | | | | |
| Buffering | Na₃PO₄ + | Na₃PO₄ + | Na₃PO₄ + | Na₃PO₄ + | Na₂HPO₄ + | Na₂HPO₄ + | Na₂HPO₄ + |
| System | Citric acid | Citric acid | Citric acid | Citric acid | NaH₂PO₄ | NaH₂PO₄ | NaH₂PO₄ |
| Flavoring | — | Peppermint | Peppermint | Peppermint | Peppermint | Peppermint | Peppermint |
| Agents(s) | | oil blend | oil blend | oil blend | oil + | oil + | oil + |
| | | | | | Menthol | Menthol | Menthol |
| | | | | | Crystals | Crystals | Crystals |
| Sweetener | — | Sucralose | Sucralose | Sucralose | Sucralose | Sucralose | Sucralose |
| Source of | — | — | Sodium | — | Sodium | — | Sodium |
| Fluoride | | | Fluoride | | Fluoride | | Fluoride |
| Dispersant | — | — | — | Polysorbate | — | — | — |
| | | | | 20 | | | |
| Aliphatic | — | — | — | — | Sodium | Sodium | Sodium |
| anionic | | | | | Lauroyl | Lauroyl | Myristoyl |
| compound | | | | | Sarcosinate | Sarcosinate | Sarcosinate |
| Thickening | — | — | — | — | Cellulose | Cellulose | Cellulose |
| Agent | | | | | Gum | Gum | Gum |
| Coloring | — | — | — | — | Titanium | Titanium | Titanium |
| Agent | | | | | Dioxide | Dioxide | Dioxide |
| (whitening) | | | | | | | |
| Abrasive | — | — | — | — | Hydrated | Hydrated | Hydrated |
| Agent | | | | | Silica | Silica | Silica |
| Water | Water | Water | Water | Water | Water | Water | Water |

Note:
Na₃PO₄: Trisodium phosphate. Na₂HPO₄: Disodium hydrogen phosphate. NaH₂PO₄: Sodium dihydrogen phosphate.

TABLE 2
Oral Care Compositions

| | Oral | Oral | Oral | Oral | Oral | Toothpaste | Toothpaste | Toothpaste |
|---|---|---|---|---|---|---|---|---|
| Ingredient | Rinse-I | Rinse-II | Rinse-III | Spray-X | Spray-Y | C | D | K |
| Stabilized source | 2.0 | 2.0 | 2.2 | 2.2 | 2.2 | 2.7 | 2.7 | 2.7 |
| of chlorine | | | | | | | | |
| dioxide | | | | | | | | |
| (5%) (i.e., | | | | | | | | |
| Chlorite Ion | | | | | | | | |
| Source only) | | | | | | | | |
| Na₃PO₄ | 0.2 | 0.3 | 0.4 | 0.2 | 0.2 | — | — | — |
| Citric Acid | 0.08 | 0.09 | 0.11 | 0.08 | 0.08 | — | — | — |
| Peppermint Oil | — | 0.08 | 0.07 | 0.75 | 0.75 | 0.76 | 0.76 | 0.76 |
| Sucralose | — | 0.01 | 0.02 | 0.04 | 0.04 | 0.2 | 0.2 | 0.2 |
| Sodium Fluoride | — | — | 0.53 | — | — | 0.24 | — | 0.21 |
| Polysorbate 20 | — | — | — | 4.0 | 1.0 | — | — | — |
| Titanium | — | — | — | — | — | 0.95 | 0.95 | 0.95 |
| Dioxide | | | | | | | | |
| Cellulose Gum | — | — | — | — | — | 1.90 | 1.90 | 1.90 |
| Hydrated Silica | — | — | — | — | — | 26.0 | 26.0 | 26.0 |
| Menthol Crystals | — | — | — | — | — | 0.14 | 0.14 | 0.14 |
| NaH₂PO₄ | — | — | — | — | — | 0.6 | 0.6 | 0.6 |
| Na₂HPO₄ | — | — | — | — | — | 1.8 | 1.8 | 1.8 |
| Sodium Lauroyl | — | — | — | — | — | 2.5 | 2.5 | — |
| Sarcosinate | | | | | | | | |
| Sodium | — | — | — | — | — | — | — | 0.25 |
| Myristoyl | | | | | | | | |
| Sarcosinate | | | | | | | | |
| Water | 97.72 | 97.52 | 96.67 | 92.73 | 95.73 | 62.21 | 62.45 | 64.49 |

Note:
Na₃PO₄: Trisodium phosphate. Na₂HPO₄: Disodium hydrogen phosphate. NaH₂PO₄: Sodium dihydrogen phosphate.

### EXAMPLE 2

### Stability Testing of Oral Rinse Compositions

Oral Rinses I, II, and III were prepared following the teaching as described herein and according to Exemplary Composition I. Stability testing of Oral Rinse I, Oral Rinse II, and Oral Rinse III were performed at room temperature (25°+2° C.). The results are summarized in Table

TABLE 3
Stability of Oral Rinses at Room Temperature (25° C. ± 2° C.)

| | Stabilized source of | | % |
|---|---|---|---|
| | chlorine dioxide (%) | | Loss in |
| Oral Rinse | Initial | 36 Months | 36 Months |
| Oral Rinse-I | 0.105 | 0.097 | 7.4 |
| Oral Rinse-II | 0.105 | 0.086 | 18.1 |
| Oral Rinse-III | 0.109 | 0.099 | 9.2 |

As Table 3 shows, only 7.4%, 18.1% and 9.2% loss of stabilized source of chlorine dioxide in 36 months at room temperature (25°+2° C.) was observed for Oral Rinse I, Oral Rinse II, and Oral Rinse III, respectively. Therefore, Oral Rinse I, Oral Rinse II, and Oral Rinse III are stable as defined herein.

### EXAMPLE 3

### Accelerated Stability Testing of Oral Spray-X

Oral Spray-X was prepared following the teaching as described herein and according to Exemplary Composition II. Accelerated stability testing of Oral Spray-X was performed at 40°+2° C. and 70-75% relative humidity (“RH”). Oral Spray-X was stored in upright position during the testing period. The results are summarized in Table 4. Accelerated stability testing at 40° C.±2° C. and 75%±5% RH is a standard accelerated stability test conducted in the pharmaceutical and cosmetic industries (Guidance for Industry: Q1A(R2) Stability Testing of New Drug Substances and Products, FDA, Revision 3 November 2003). The stability testing of Oral Spray-X adhered to accepted norms of prior art and the pharmaceutical industry.

TABLE 4
Accelerated stability of Oral Spray-X at 40° ± 1° C and 70-75% RH

| | Initial | 1 Month | | 2 Months | | 3 Months | | 6 Months | |
|---|---|---|---|---|---|---|---|---|---|
| | SCD* | SCD | Loss | SCD | Loss | SCD | Loss | SCD | Loss |
| Composition | (%) | (%) | (%) | (%) | (%) | (%) | (%) | (%) | (%) |
| Oral Spray-X | 0.110 | 0.104 | 5.5 | 0.105 | 4.6 | 0.095 | 13.6 | 0.096 | 12.3 |

*SCD: Stabilized source of chlorine dioxide

As Table 4 shows, only 5.5%, 4.6%, 13.6%, and 12.3% loss of stabilized source of chlorine dioxide in Oral Spray-X was observed at 40°+1° C. and 70-75% RH in 1, 2, 3, and 6-month time points, respectively. Measurement variability in estimation of chlorine dioxide by titration method is up to 10%. Therefore, the observed variation in % loss is attributed to variation in the method of measurement. Therefore, Oral Rinse I, Oral Rinse II, and Oral Rinse III are stable as defined herein. As defined here in the stability at 40°+2° C. and 75%±5% RH for 6 months is equivalent to 2 years of shelf life at room temperature. The results in Table 4 demonstrated that Oral Spray-X has a shelf life of at least 24 months (2 years) at room temperature. Stabilized source of chlorine dioxide and buffering system was same in Oral Spray-X and Oral Spray-Y Therefore, similar stability was anticipated for Oral Spray-Y.

### EXAMPLE 4

### Antiviral Activity Against COVID-19 Virus

The virucidal efficacy suspension test was performed to determine the antiviral activity of Oral Rinse-I against COVID-19 virus. The test method used was ASTM E1052-20 (see www.astm. org/Standards/E1052.htm), herein incorporated by reference, recommended by ASTM International formerly known as American Society for Testing Materials. This is a Standard Validated Test Method to Assess the Activity of Microbicides against Viruses in Suspension.

Test Conditions:
1. Test virus: Severe Acute Respiratory Syndrome-related Coronavirus 2 (SARS-CoV-2 or COVID-19 Virus), Strain: USA-WA1/2020.
2. Host: Vero E6 cells, ATCC CRL-1586
3. Test Composition: Oral Rinse-I, as described in TABLE 2, above
4. Dilution medium: Minimum Essential Medium (MEM)+2% Newborn Calf Serum (NCS)
5. Neutralizer: MEM+10% NCS+0.5% Sodium thiosulphate
6. Contact times: 30 and 60 seconds
7. Contact temperature: 21° C.

Test Procedure: Indicator cells Vero-E6 cells were obtained from American Type Culture Collection (ATCC) and maintained in cell culture at 36±2° C. with 5±3% CO₂ prior to seeding. The indicator cell plates were prepared between 12 and 30 hours priorto inoculation with test sample. The cells were seeded in 24-well plates at a density of 1×10⁵ cells/mL at 1.0 mL per well. The original stock virus (SARS-CoV-2) used contained 5.0% fetal bovine serum (FBS). The controls and test parameters are summarized in Table 5.

TABLE 5
Controls and Test Parameters for Antiviral Test of COVID-19 Virus

| Parameter | Summary | Plate Replicates |
|---|---|---|
| Virucidal Efficacy | Virus + Test Product − Exposure − | 8 per group |
| suspension test | Neutralization − Dilution − Plating | |
| Virus Control | Virus + Diluent − Neutralization − Dilution − Plating | 8 per group |
| Cytotoxicity | Test Product + Diluent − Neutralization − | 8 per group |
| Control | Dilution − Plating | |
| Neutralization | Test Product + Diluent − Neutralization − Virus | 8 per group |
| Effectiveness/Viral | inoculation − Dilution − Plating | |
| Interference Control | | |
| Cell Viability/Media | Maintenance medium | 8 per group |
| Sterility Control | | |

Virus Suspension Test: Two replicates at each contact time exposure were performed. A 0.3 mL aliquot of COVID-19 virus (SARS-CoV-2 test virus) was transferred to a vial containing 2.7 mL of Oral Rinse-I (test composition, as described in TABLE 2 herein). The challenge suspension was exposed to the test composition for the contact time. Immediately after the contact exposure, the 3.0 mL aliquot of the test virus/product suspension was neutralized with 3.0 mL of neutralizer, mixed thoroughly, and serially diluted in Dilution Medium (DM). Each dilution was plated in eight replicates

Virus Control: Two replicates of the Virus Control were performed. A 0.3 mL aliquot of the test virus was added to 2.7 mL of DM and exposed for the contact time at test temperature. Immediately after the contact exposure, a 3.0 mL aliquot of the test virus/product suspension was neutralized with 3.0 mL of neutralizer, mixed thoroughly, and serially diluted in Dilution Medium (DM). Each dilution was plated in eight replicates.

Neutralization Effectiveness/Viral Interference Control: A 0.3 mL aliquot of DM was added to a vial containing a 2.7 mL aliquot of the test composition, mixed by vortexing (i.e., mixing) and held for the contact time. Upon completion of the contact time, an aliquot or the entirety of the reaction mixture was immediately mixed with an equal volume of neutralizer via vortexing (3.0 mL). Subsequent serial dilutions of this mixture were made in DM. An aliquot of the virus was added to each dilution and thoroughly mixed. 100 μL of low tittered virus was added to 4.5 mL of each dilution and held for a period of no shorter than the longest contact time. Selected dilutions were inoculated onto the host cell plates in eight replicates.

Cytotoxicity Control: This control was performed for each test composition at one replicate and one contact time (the longer of the two). Selected dilutions of the sample obtained from the NE/VI control were inoculated onto host cells in eight replicates without any virus to determine any cytotoxic effects from the test composition.

Cell Viability/Media Sterility Control: Intact cell culture served as the control of cell culture viability. Dilution Medium was added to all cell control wells. All plates were incubated in a CO₂ incubator for 7 days at the appropriate temperature for the virus. Cytopathic/cytotoxic effects were monitored using an Inverted Compound Microscope.

Test Acceptance Criteria: The test will be acceptable for evaluation of the test results if the criteria listed below are satisfied.
1 Virus must be recovered from the neutralizer effectiveness/viral interference control (not exhibiting cytotoxicity).
2 Viral-induced cytopathic effects (CPE) must be distinguishable from test composition induced toxicity.
3 The cell viability control must remain viable throughout the course of the assay period and exhibit absence of virus.

Calculations
The 50% Tissue Culture Infectious Dose per mL (TCID₅₀/mL) was determined using the Spearman-Karber method using the following formula:

m=xₖ+(d/2)−dΣpᵢ

where:
m=the logarithm of the dilution at which half of the wells are infected relative to the test volume
xₖ=the logarithm of the smallest dosage which induces infection in all cultures
d=the logarithm of the dilution factor
pᵢ=the proportion of positive results at dilution i
Σpᵢ=the sum of pᵢ (starting with the highest dilution producing 100% infection)

The values were converted to TCID50/mL using a sample inoculum of 0.05 mL.

The Viral Load was Determined in the Following Manner:

Viral Load (Log₁₀TCID50)=Titer (Log₁₀ TCID₅₀/mL)+Log₁₀[Volume (mL)×Volume Correction](e.g., neutralization)

Note: The volume (mL) of the Undiluted (10°) sample was used in the above equation

The Log₁₀ Reduction Factor (LRF) was calculated in the following manner:

LRF=Initial Viral Load (Log₁₀TCID₅₀)−Output Viral Load (Log₁₀ TCID₅₀)

The Average Logio Virus Recovery Control was calculated in the following manner: Average=(Viral Load of Virus Recovery Control Replicate 1+Viral Load of Virus Recovery Control Replicate 2) /2

### Results

Virus was detected in all inoculated wells of Neutralizer Effectiveness/Viral Interference (NE/VI) control at 10⁻¹, 10⁻² and 10⁻³ dilutions and at 60 seconds contact time. Therefore, the results for NE/VI controls were acceptable.

Virus was not detected in all inoculated wells of Cytotoxicity control at 10⁻¹, 10⁻² and 10⁻³ dilutions and at 60 seconds contact time. Therefore, the results for cytotoxicity control were acceptable.

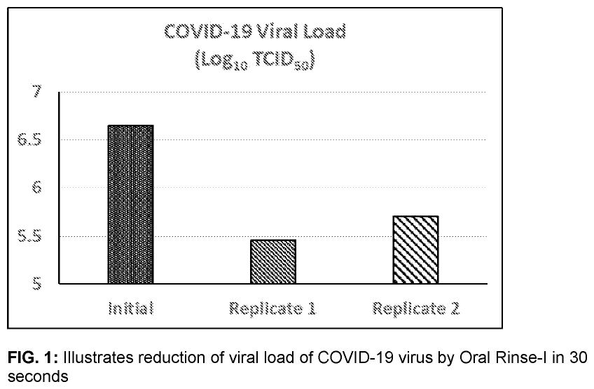

The results for antiviral activity of Oral Rinse-I, as described in TABLE 2 herein, towards COVID-19 virus are summarized in Table 6 and FIG. 1.

TABLE 6
Reduction of COVID-19 virus by Oral Rinse-I

| | | Initial Viral | Output Viral | | |
|---|---|---|---|---|---|
| Contact | | Load | Load | Log₁₀ | % |
| Time | Replicate | (Log₁₀ TCID₅₀)* | (Log₁₀ TCID₅₀) | Reduction | Reduction |
| 30 Seconds | 1 | 6.65 | 5.46 | 1.19 | 93.543 |
| | 2 | 6.65 | 5.71 | 0.94 | 88.518 |
| 60 Seconds | 1 | 6.65 | 5.71 | 0.94 | 88.518 |
| | 2 | 6.65 | 5.83 | 0.82 | 84.864 |

*The average VRC for the corresponding contact time was used as initial viral load

The results confirmed that all controls met the criteria for a valid test. Oral Rinse-I did not show any cytotoxicity to host cells. The data suggest that Oral Rinse-I reduced from 88.5% to 93.5% of the initial viral load of the COVID-19 virus within 30 seconds after first contact. Further exposure to 60 seconds did not increase the antiviral activity. Therefore, Oral Rinse-I is effective in reducing the viral load of COVID-19 virus by about 90% of the initial viral load in 30 seconds of contact time at room temperature (21° C.). As described herein, one of ordinary skill in the art would know that reduction in initial viral load of a virus is also referred as elimination of-, reducing infectivity of-, reducing viral count of-, destruction of- or killing of the virus. Antiviral activity of Oral Rinse-I is thought to be a result of oxidation of biomolecules such as proteins and lipids on the COVID-19 virus by chlorine dioxide released from Oral Rinse-I within 30 seconds or less of its contact with the virus. The formulation and dosage of the stabilized source of chlorine dioxide and buffering system is the same in Oral Rinse-I, Oral Rinse-II, Oral Rinse-III, Oral Spray-X, Oral Spray-Y, Toothpaste C, Toothpaste D and Toothpaste K as all compositions are described herein. Therefore, similar antiviral activity against COVID-19 virus is expected with Oral Rinse-II, Oral Rinse-III, Oral Spray-X or Oral Spray-Y, Toothpaste C, Toothpaste D or Toothpaste K.

### EXAMPLE 5

### Antiviral Activity Against COVID-19 Virus

The extent of reduction of viral load of COVID-19 virus thereby reducing its infectivity in vitro by Oral Rinse-I, Oral Rinse-II, Oral Spray-X and Toothpaste C was investigated. In particular, the ability of Oral Rinse-I, Oral Rinse-II, Oral Spray-X and Toothpaste C as described herein, to control the replication of SARS-CoV-2 in vitro was studied.

Test Conditions:
1. Test virus: Acute Respiratory Syndrome-related Coronavirus 2 (SARS-CoV-2 or COVID-19 Virus) strain USA-WA1/2020, acquired from BEI Resources (NR-52281).
2. Host: Vero E6 cells, ATCC CRL-1586
3. Test Compositions (as discussed in this Example 5 only): Oral Rinse-I, Oral Rinse-II,

Oral Spray-X or Toothpaste C as described in TABLE 2, above
4. Contact times: 30- and 60-seconds for oral rinse and oral spray compositions and 30-, 60-, and 120-seconds for Toothpaste C. The selected contact times represent normal time of use of respective composition during regular home oral care practice.

Test Procedures:

Oral Rinse-I, Oral Rinse-II, and Oral Spray-X were tested individually in separate experiments following same protocol for investigating their ability to reduce viral load of SARS-CoV-2. 2.7 ml of Oral Rinse-I, Oral Rinse-II, or Oral Spray-X (Test Composition, as discussed in this Example 5 only) was mixed with 0.3 mL of COVID-19 virus suspension in a 15 mL conical tube and incubated for 30 or 60 seconds. After the designated time, 3.0 mL of 0.5% sodium thiosulfate in water was added to the conical vial. This was done once on one day and twice on a second day for a total of three replicates. A PBS control, using 2.7 mL PBS in place of the 2.7 mL Test Composition was also conducted once on one day and twice on a second day. Liquid from the conical tubes was diluted and added to Vero E6 cells. Plates were examined at 3 or 4 days post-for cytopathic effect (CPE). In addition, a cytotoxicity/neutralization control was performed with 2.7 mL of Test Composition and 0.3 mL of PBS rather than virus. This was split, adding the material directly to Vero cells as a cytotoxicity control and adding virus to dilutions of the liquid prior to the addition to Vero cells. The addition of virus to the neutralized solution without virus was to demonstrate that the Test Composition had indeed been neutralized after the addition of the 0.5% sodium thiosulfate and would not prevent viral induced CPE in Vero cells.

Vero E6 cells were cultured in growth media consisting of Dulbeco's Modified Eagle Medium/F12 supplemented with 5% FBS (Fetal Bovine Serum), and PSN (penicillin, streptomycin, and neomycin).

The Vero E6 cells were plated on 96-well plates one to three days before the assay and were allowed to grow to ˜60-70% confluence. On the day of the assay, we prepared 2.7 ml of Test Composition in a 15 mL conical tube and added 0.3 ml of COVID-19 virus strain USA-WA1/2020 to the tube and incubated for 30 or 60 seconds. After the designated time, 3.0 mL of 0.5% sodium thiosulfate in water was added to the conical vial. A PBS control, using 2.7 mL PBS in place of the 2.7 mL Test Composition was also conducted. One replicate was conducted on one test day and two additional replicates were performed on a second test day. In addition, a cytotoxicity/neutralization control was performed with 2.7 mL of Test Composition and 0.3 mL of PBS rather than virus. This was split, adding the material directly to Vero cells as a cytotoxicity control and adding virus to dilutions of the liquid prior to the addition to Vero cells. The addition of virus to the neutralized solution without virus was to demonstrate that Test Composition had indeed been neutralized after the addition of the 0.5% sodium thiosulfate and would not prevent viral induced CPE in Vero cells. The cytotoxicity/neutralization control was performed once for each time point (30 or 60 seconds) on the second day of testing. Samples were added to an empty 96 well plate and diluted 1:10 down the plate in DMEM/F12. These dilutions were then transferred to a plate of Vero cells with media removed. After approximately 45 minutes, DMEM/F12 supplemented with FBS was added to cells to feed them for the next 3-6 days. This incubation period of approximately 45 minutes is to allow the virus to adsorb to cells without interference from FBS. Cytotoxicity controls of the test articles without virus added were also performed. The assay was executed in five replicates for each condition.

After 3 or 4 days, cells were examined for the presence of cytopathic effect (CPE) associated with viral presence and replication. Examination is done using a microscope (10× objective to view the entire well at once) and observing the morphology of the cells. Healthy Vero cells have somewhat transparent appearance with pinched or rounded ends in a monolayer of cells with little to no space between cells. Dead cells displaying CPE are often not adhered to the plate, round and much smaller than living cells. Furthermore, the healthy Vero cells cover much of the surface of the well but wells containing cells with CPE have areas of the well where no cells are adherent, described as empty space. Any well displaying CPE is marked as positive whether the whole well is affected or only a small patch is indicative of viable virus present.

Test procedure for Toothpaste C: Test procedure for Toothpaste C was similar to that described for oral rinse and oral spray compositions above in this Example except for the sample preparation and volumes used for virus treatment. A slurry of the toothpaste was made by adding 4 g toothpaste to 8 ml of phosphate buffered saline (PBS) and vortexing (i.e., 1→3 dilution). This slurry was centrifuged to pellet large particulates and syringe filtered through a 0.22 μm filter to remove smaller debris. This filtered clear supernatant was treated as an undiluted sample. 900 μL of supernatant of the toothpaste slurry (toothpaste solution) was mixed with 100 μL virus stock (3.16E6 TCID_{50/}ml) and allowed a contact time of 30-, 60- or 120-seconds. After the contact time was reached, 1 mL of 0.5% sodium thiosulfate was added to each sample to neutralize the reaction. Similar volumes were used for all controls.

Results were calculated using the Reed & Muench Calculator (produced by BD Lindenbach from “Measuring HCV infectivity produced in cell culture and in vivo” Methods Mol Biol. (2009) 510:329-36). Results are shown as Log reduction relative to timed controls as well as a percent reduction of viral load of COVID-19 virus.

Results

Cytotoxicity was observed at the 1:10 diluted Oral Rinse-I and Oral Rinse-II test. Similarly, cytotoxicity was observed at 1:10 and 1:100 diluted Oral Spray-X. This did not confound the assay, as data for these dilutions in Oral Rinse-I, Oral Rinse-II or Oral Spray-X were not intended to be used in the calculation of viral titers. The viral titers used affected further dilutions on the plate. Because the calculation of the TCID50 is primarily an endpoint titer calculation, the rows between no CPE and CPE are the most important for interpreting the results. All neutralization tests showed CPE, indicating that sodium thiosulfate neutralized the Oral Rinse-I, Oral Rinse-II or Oral Spray-X. Day 3 or 4 reads showed CPE in wells from Oral Rinse-I, Oral Rinse-II or Oral Spray-X treated samples, however there was less than that in PBS control samples. Control samples in Toothpaste C test experiment showed a 0.74 log loss of virus compared to the back titer. Since the control samples are treated identically to the toothpaste test samples (without the test article), this determines non-specific loss of the virus in the assay. The Toothpaste C solution (1→3 dilution) alone or with 0.5% sodium thiosulfate showed cytotoxicity at the same levels, indicating that 0.5% sodium thiosulfate did not effectively neutralize the product. However, observed cytotoxicity did not affect the result interpretation and conclusions. All uninfected controls remained healthy and did not display any CPE at the end of the observation period.

The results are summarized in Tables 7 through 11.

TABLE 7
Reduction of COVID-19 virus by Oral Rinse-I

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{†} | TCID₅₀) | TCID₅₀) | Reduction | Reduction |
| 30 | 1 | 5.79 | 5.83 | 3.83 | 1.96 | 98.4 |
| Seconds | 2 | | 3.50 | | | |
| | 3 | | 2.17 | | | |
| 60 | 1 | 5.31 | 4.50 | 3.92 | 1.39 | 96.3 |
| Seconds | 2 | | 3.63 | | | |
| | 3 | | 3.63 | | | |

^{†}The mean viral recovery control value for the corresponding contact time was used as the Initial Load.

TABLE 8
Reduction of COVID-19 virus by Oral Rinse-II

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{†} | TCID₅₀) | TCID₅₀) | Reduction | Reduction |
| 30 | 1 | 6.50 | 4.63 | 4.69 | 1.81 | 98.4 |
| Seconds | 2 | | 4.63 | | | |
| | 3 | | 4.83 | | | |
| 60 | 1 | 6.17 | 5.38 | 4.46 | 1.71 | 98.0 |
| Seconds | 2 | | 3.50 | | | |
| | 3 | | 4.50 | | | |

^{†}The mean viral recovery control value for the corresponding contact time was used as the Initial Load.

TABLE 9
Reduction of COVID-19 virus by Oral Spray-X

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{†} | TCID₅₀) | TCID₅₀) | Reduction | Reduction |
| 30 | 1 | 6.50 | 3.50 | 3.52 | 2.98 | 99.9 |
| Seconds | 2 | | 3.56 | | | |
| | 3 | | 3.50 | | | |
| 60 | 1 | 6.17 | 3.50 | 3.50 | 2.67 | 99.7 |
| Seconds | 2 | | 3.50 | | | |
| | 3 | | 3.50 | | | |

^{†}The mean viral recovery control value for the corresponding contact time was used as the Initial Load.

TABLE 10
Reduction of COVID-19 virus by Toothpaste C*

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{†} | TCID₅₀) | TCID₅₀) | Reduction | Reduction |
| 30 | 1 | 5.76 | 3.50 | 3.50 | 2.26 | 99.46 |
| Seconds | 2 | | 3.50 | | | |
| | 3 | | 3.50 | | | |
| 60 | 1 | 5.76 | 3.50 | 3.50 | 2.26 | 99.46 |
| Seconds | 2 | | 3.50 | | | |
| | 3 | | 3.50 | | | |
| 120 | 1 | 5.76 | 3.50 | 3.50 | 2.26 | 99.46 |
| Seconds | 2 | | 3.50 | | | |
| | 3 | | 3.50 | | | |

*Supernatant of 1-3 diluted toothpaste slurry. The supernatant was treated as an undiluted sample.
^{†}The mean viral recovery control value for the corresponding contact time was used as the Initial Load.

TABLE 11
Summary of Reduction of COVID-19 virus by Oral Rinse-I,
Oral Rinse-II, Oral Spray-X and Toothpaste C

| | Reduction in Viral Load of SARS-CoV-2 | | |
|---|---|---|---|
| Composition | 30 Seconds | 60 Seconds | 120 Seconds |
| Oral Rinse-I | 98.4% | 96.3% | Not Tested |
| Oral Rinse-II | 98.4% | 98.0% | Not Tested |
| Oral Spray-X | 99.9% | 99.7% | Not Tested |
| Toothpaste C* | 99.46% | 99.46% | 99.46% |

*Supernatant of 1-3 diluted toothpaste slurry. The supernatant was treated as an undiluted sample.

The results confirmed that all controls met the criteria for a valid test. The data showed that Oral Rinse-I, Oral Rinse-II, Oral Spray-X and Toothpaste C reduced 98.4%, 98.4%, 99.9% and 99.46% of the initial viral load of the COVID-19 virus within 30 seconds of contact, respectively. The reduction of viral load reduced the infectivity of CODID-19 virus by 98.4%, 98.4%, 99.9% and 99.46% by Oral Rinse-I, Oral Rinse-II, Oral Spray-X and Toothpaste C in 30 seconds, respectively. There did not appear to be a substantive difference between 30- and 60-seconds of contact time for oral rinse and oral spray compositions. The results were same at 30-, 60- and 120-seconds of contact time for Toothpaste C. As described herein, one of ordinary skill in the art would know that reduction in initial viral load of a virus is also referred as elimination of-, reducing infectivity of-, reducing viral count of-, destruction of- or killing of the virus. As described herein antiviral activity of Oral Rinse-I, Oral Rinse-II, Oral Spray-X or Toothpaste C is thought to be a result of oxidation of biomolecules such as proteins and lipids on the COVID-19 virus by chlorine dioxide released from respective composition within 30 seconds or less of its contact with the virus. The formulation and dosage of the stabilized source of chlorine dioxide is the same in Oral Rinse-I, Oral Rinse-II, Oral Rinse-III, Oral Spray-X, Oral Spray-Y, Toothpaste C, Toothpaste D and Toothpaste K as all compositions are described herein. Therefore, similar antiviral activity against COVID-19 virus is expected with Oral Rinse-III, Oral Spray-Y, Toothpaste D or Toothpaste K.

### EXAMPLE 6

### Antiviral Activity Against SARS Virus

The virucidal efficacy suspension test was performed to determine the antiviral activity of Oral Rinse-I against SARS virus (SARS-CoV virus). The test method used was ASTM E1 052-20 (see www.astm.org/Standards/E1052.htm) recommended by ASTM International formerly known as American Society for Testing Materials This is a Standard Validated Test Method to Assess the Activity of Microbicides against Viruses in Suspension.

Test Conditions:
1. Test virus: Severe Acute Respiratory Syndrome-related Coronavirus (SARS-CoV or SARS virus), Strain: CDC 200300592.
2. Host: Vero E6 cells, ATCC CRL-1586
3. Test Composition: Oral Rinse-I, as described in TABLE 2, above
4. Dilution medium: Minimum Essential Medium (MEM)+2% Newborn Calf Serum (NCS)
5. Neutralizer: MEM+10% NCS+0.5% Sodium thiosulphate
6. Contact times: 30 and 60 seconds
7. Contact temperature: 21° C.

Test Procedure: Indicator cells Vero-E6 cells were obtained from American Type Culture Collection (ATCC) and maintained in cell culture at 36±2° C. with 5±3% CO₂ prior to seeding. The indicator cell plates were prepared 12-30 hours priorto inoculation with test sample. The cells were seeded in 96-well plates at a density of 8×10⁴ cells/mL at 0.15 mL per well. The original stock virus (SARS-CoV) used contained 5.0% FBS serum. The controls and test parameters are summarized in Table 12.

TABLE 12
Controls and Test Parameters for Antiviral Test of SARS Virus

| Parameter | Summary | Plate Replicates |
|---|---|---|
| Virucidal Efficacy | Virus + Test Product − Exposure − | 8 per group |
| suspension test | Neutralization − Dilution − Plating | |
| Virus Control | Virus + Diluent − Neutralization − Dilution − Plating | 8 per group |
| Cytotoxicity | Test Product + Diluent − Neutralization − | 8 per group |
| Control | Dilution − Plating | |
| Neutralization | Test Product + Diluent − Neutralization − Virus | 8 per group |
| Effectiveness/Viral | inoculation − Dilution − Plating | |
| Interference Control | | |
| Cell Viability/Media | Maintenance medium | 8 per group |
| Sterility Control | | |

Virus Suspension Test: Two replicates at each contact time exposure were performed. A 0.3 mL aliquot of SARS virus (test virus) was transferred to a vial containing 2.7 mL of Oral Rinse-I (test composition). The challenge suspension was exposed to the test solution for the contact time. Immediately after the contact exposure, the 3.0 mL aliquot of the test virus/product suspension was neutralized with 3.0 mL of neutralizer, mixed thoroughly, and serially diluted in Dilution Medium (Dlvi). Each dilution was plated in eight replicates.

Virus Control: Two replicates of the Virus Control were performed. A 0.3 mL aliquot of the test virus was added to 2.7 mL of DM and exposed for the contact time at test temperature. Immediately after the contact exposure, a 3.0 mL aliquot of the test virus/product suspension was neutralized with 3.0 mL of neutralizer, mixed thoroughly, and serially diluted in Dilution Medium (DM). Each dilution was plated in eight replicates.

Neutralization Effectiveness/Viral Interference Control: A 0.3 mL aliquot of DM was added to a vial containing a 2.7 mL aliquot of the test composition, mixed by vortexing and held for the contact time. Upon completion of the contact time, an aliquot or the entirety of the reaction mixture was immediately mixed with an equal volume of neutralizer via vortexing (3.0 mL). Subsequent serial dilutions of this mixture were made in DM. An aliquot of the virus was added to each dilution and thoroughly mixed. 100 μL of low tittered virus was added to 4.5 mL of each dilution and held for a period of no shorter than the longest contact time. Selected dilutions were inoculated onto the host cell plates in eight replicates.

Cytotoxicity Control: This control was performed for each test composition at one replicate and one contact time (the longer of the two). Selected dilutions of the sample obtained from the NE/VI control were inoculated onto host cells in eight replicates without any virus to determine any cytotoxic effects from the test composition.

Cell Viability/Media Sterility Control: Intact cell culture served as the control of cell culture viability. Dilution Medium was added to all cell control wells. All plates were incubated in a CO₂ incubator for 7 days at the appropriate temperature for the virus. Cytopathic/cytotoxic effects were monitored using an Inverted Compound Microscope.

Test Acceptance Criteria: The test will be acceptable for evaluation of the test results if the criteria listed below are satisfied.
1 Virus must be recovered from the neutralizer effectiveness/viral interference control (not exhibiting cytotoxicity).
2 Viral-induced cytopathic effects (CPE) must be distinguishable from test composition induced toxicity.
3 The cell viability control must remain viable throughout the course of the assay period and exhibit absence of virus.

Calculations

The 50% Tissue Culture Infectious Dose per mL (TCID50/mL) was determined using the Spearman-Karber method using the following formula:

m=xₖ+(d/2)−d Σpᵢ

where:
m=the logarithm of the dilution at which half of the wells are infected relative to the test volume
xₖ=the logarithm of the smallest dosage which induces infection in all cultures
d=the logarithm of the dilution factor
pᵢ=the proportion of positive results at dilution i
Σpᵢ=the sum of pᵢ (starting with the highest dilution producing 100% infection)
The values were converted to TCID5o/mL using a sample inoculum of 0.05 mL.

The Viral Load was Determined in the Following Manner:

Viral Load (Log₁₀TCID₅₀)=Titer (Log₁₀TCID₅₀/mL)+Log₁₀ [Volume (mL)×Volume Correction] (e.g., neutralization)

Note: The volume (mL) of the Undiluted (10°) sample was used in the above equation

The Log₁₀ Reduction Factor (LRF) was Calculated in the Following Manner:

LRF=Initial Viral Load (Log₁₀TCID₅₀)−Output Viral Load (Log₁₀ TCID₅₀)

The Average Log₁₀ Virus Recovery Control was Calculated in the Following Manner:

Average=(Viral Load of Virus Recovery Control Replicate 1+Viral Load of Virus Recovery Control Replicate 2)/2

Results

Virus was detected in all inoculated wells of Neutralizer Effectiveness/Viral Interference (NE/VI) control at 10⁻¹, 10⁻² and 10⁻³ dilutions and at 60 seconds contact time. Therefore, the results for NE/VI controls were acceptable.

Virus was not detected in all inoculated wells of Cytotoxicity control at 10⁻¹, 10⁻² and 10⁻³ dilutions and at 60 seconds contact time. Therefore, the results for cytotoxicity control were acceptable.

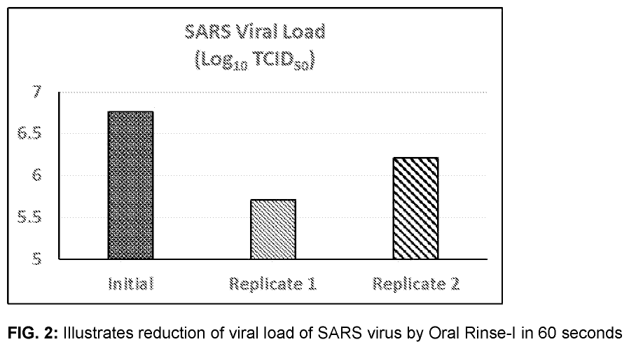

The results for antiviral activity of Oral Rinse-I towards SARS virus are summarized in Table 13 and FIG. 2.

TABLE 13
Reduction of SARS virus by Oral Rinse-I

| | | Initial Viral | Output Viral | | |
|---|---|---|---|---|---|
| Contact | | Load | Load | Log₁₀ | % |
| Time | Replicate | (Log₁₀ TCID₅₀)* | (Log₁₀ TCID₅₀) | Reduction | Reduction |
| 30 Seconds | 1 | 6.77 | Uninterpretable data results | | |
| | 2 | 6.77 | 6.58 | 0.19 | 35.435 |
| 60 Seconds | 1 | 6.77 | 5.71 | 1.06 | 91.290 |
| | 2 | 6.77 | 6.21 | 0.56 | 72.458 |

*The average VRC for the corresponding contact time was used as initial viral load

The results confirmed that all controls met the criteria for a valid test. Oral Rinse-I did not show any cytotoxicity to host cells. The data suggest that Oral Rinse-I reduced 35.4% of the initial viral load of the SARS virus within 30 seconds. Further exposure to 60 seconds increased the antiviral activity. Oral Rinse-I reduced from 72.46% to 91.23% of the initial viral load of the SARS virus within 60 seconds. Therefore, Oral Rinse-I is effective in reducing the viral load of SARS virus up to about 91% of the initial viral load of the SARS virus within 60 seconds of contact time at room temperature (21° C.). As described herein, one of ordinary skill in the art would know that reduction in initial viral load of a virus is also referred as elimination of-, reducing infectivity of-, reducing viral count of-, destruction of- or killing of the virus. Antiviral activity of Oral Rinse-I is thought to be a result of oxidation of biomolecules such as proteins and lipids on the SARS virus by chlorine dioxide released from Oral Rinse-I within seconds of its contact with the virus. The formulation and dosage of stabilized source of chlorine dioxide is same in Oral Rinse-I, Oral Rinse-II, Oral Rinse-III, Oral Spray-X, Oral Spray-Y, Toothpaste C, Toothpaste D and Toothpaste K. Therefore, similar antiviral activity against SARS virus is expected with Oral Rinse-II, Oral Rinse-III, Oral Spray-X, Oral Spray-Y, Toothpaste C, Toothpaste D or Toothpaste K.

### EXAMPLE 7

### Antiviral Activity Against Influenza A Virus

Antiviral activity of Oral Rinse-I, Oral Rinse-II, Oral Spray-X and Toothpaste C against Influenza A virus was tested using a virucidal efficacy suspension test. The test method used was ASTM E1052-20 (see www.astm.org/Standards/E1052.htm) as recommended by ASTM International, formerly known as American Society for Testing Materials. This is a Standard Validated Test Method to Assess the Activity of Microbicides against Viruses in Suspension.

Test Conditions:
1. Test virus: Influenza A virus (H3N2), A/Hong Kong/8/68 procured from Charles River Laboratories.
2. Host: MDCK cells, ATCC CCL-34
3. Test Composition: Oral Rinse-I, Oral Rinse-II, Oral Spray-X and Toothpaste C as described in TABLE 2, above
4. Dilution medium: Minimum Essential Medium (MEM)+1.0 μg/mL Trypsin
5. Neutralizer: MEM+1% NCS+0.5% Sodium thiosulphate
6. Contact times: 30- and 60-seconds for oral rinse and oral spray compositions and 30-, 60- and 120-seconds for the toothpaste composition.
7. Contact temperature: 21° C.

Test Procedure:

MDCK cells were obtained from ATCC and maintained in cell culture at 36±2° C. with 5±3% CO₂ prior to seeding. The indicator cell plates were prepared 12-30 hours priorto inoculation with test sample. The cells were seeded in 24-well plates at a density of 1×10⁵ cells/mL at 1.0 mL per well. The original stock virus used contained 0% serum. The controls and test parameters are summarized in Table 14.

TABLE 14
Controls and Test Parameters for Antiviral Test of Influenza A Virus

| Parameter | Summary | Plate Replicates |
|---|---|---|
| Virucidal Efficacy | Virus + Test Product − Exposure − | 4 per group |
| suspension test | Neutralization − Dilution − Plating | |
| Virus Control | Virus + Diluent − Neutralization − Dilution − Plating | 4 per group |
| Cytotoxicity | Test Product + Diluent − Neutralization − | 4 per group |
| Control | Dilution − Plating | |
| Neutralization | Test Product + Diluent − Neutralization − Virus | 4 per group |
| Effectiveness/Viral | inoculation − Dilution − Plating | |
| Interference Control | | |
| Cell Viability/Media | Dilution medium | 4 per group |
| Sterility Control | | |

Toothpaste C sample preparation: Toothpaste C slurry was prepared by mixing 1 part of the toothpaste with 2 parts of PBS (1→3 dilution). The slurry was vortexed and centrifuged to get supernatant free of suspended particles. The supernatant was treated as an undiluted sample.

Virus Suspension Test: Two replicates at each contact time exposure were performed. A 0.3 mL aliquot of Influenza A virus (test virus) was transferred to a vial containing 2.7 mL of the test composition and mixed by vortex. The challenge suspension was exposed to the test solution for the contact time. Immediately after the contact exposure, the 3.0 mL aliquot of the test virus/product suspension was neutralized with 3.0 mL of neutralizer, mixed thoroughly, and serially diluted in Dilution Medium (DM). Each dilution was plated in four replicates

Virus Control: Two replicates of the Virus Control were performed. A 0.3 mL aliquot of the test virus was added to 2.7 mL of DM, mixed by vortex and exposed for the contact time at test temperature Immediately after the contact exposure, a 3.0 mL aliquot of the test virus/product suspension was neutralized with 3.0 mL of neutralizer, mixed thoroughly, and serially diluted in DM. Each dilution was plated in four replicates.

Neutralization Effectiveness/Viral Interference Control: A 0.3 mL aliquot of DM was added to a vial containing a 2.7 mL aliquot of the test composition, mixed by vortexing and held for the contact time. Upon completion of the contact time, an aliquot or the entirety of the reaction mixture was immediately mixed with an equal volume of neutralizer via vortexing (3.0 mL). Subsequent serial dilutions of this mixture were made in DM. An aliquot of the virus was added to each dilution and thoroughly mixed. 100 μL of low tittered virus was added to 4.5 mL of each dilution and held for a period of no shorter than the longest contact time. Selected dilutions were inoculated onto the host cell plates in four replicates.

Cytotoxicity Control: This control was performed for each test composition at one replicate and one contact time (the longer of the two). Selected dilutions of the sample obtained from the NE/VI control were inoculated onto host cells in four replicates without any virus to determine any cytotoxic effects from the test composition.

Cell Viability/Media Sterility Control: Intact cell culture served as the control of cell culture viability. Dilution Medium was added to all cell control wells. All plates were incubated in a CO₂ incubator for 6 days at the appropriate temperature for the virus. Cytopathic/cytotoxic effects were monitored using an Inverted Compound Microscope.

Test Acceptance Criteria: Test acceptance criteria:
1 Virus must be recovered from the neutralizer effectiveness/viral interference control (not exhibiting cytotoxicity).
2 Viral-induced cytopathic effects (CPE) must be distinguishable from test composition induced toxicity.
3 The cell viability control must remain viable throughout the course of the assay period and exhibit absence of virus.

Calculations
The 50% Tissue Culture Infectious Dose per mL (TCID50/mL) was determined using the Spearman-Karber method using the following formula:

m=xₖ+(d/2)−d Σpᵢ

where:
m=the logarithm of the dilution at which half of the wells are infected relative to the test volume
xₖ=the logarithm of the smallest dosage which induces infection in all cultures
d=the logarithm of the dilution factor
pᵢ=the proportion of positive results at dilution i
Σpᵢ=the sum of pᵢ (starting with the highest dilution producing 100% infection)

The values were converted to TCID50/mL using a sample inoculum of 0.05 mL.

The Viral Load was determined in the following manner:

Viral Load (Log₁₀ TCID50)=Titer (Log₁₀ TCID₅₀/mL)+Log₁₀[Volume (mL)×Volume Correction] (e.g., neutralization)

Note: The volume (mL) of the Undiluted (10°) sample was used in the above equation The Log₁₀ Reduction Factor (LRF) was calculated in the following manner:

LRF=Initial Viral Load (Log₁₀TCID₅₀)—Output Viral Load (Log₁₀ TCID₅₀)

The Average Log₁₀ Virus Recovery Control was calculated in the following manner:

Average=(Viral Load of Virus Recovery Control Replicate 1+Viral Load of Virus Recovery Control Replicate 2)/2

Results

The results are acceptable and valid as (1) virus was recovered from neutralizer effectiveness/viral interference (NE/VI) control, (2) viral-induced cytopathic effect (CPE) was distinguishable from test substance induced toxicity and (3) cell viability control remained viable throughout the course of the assay period.

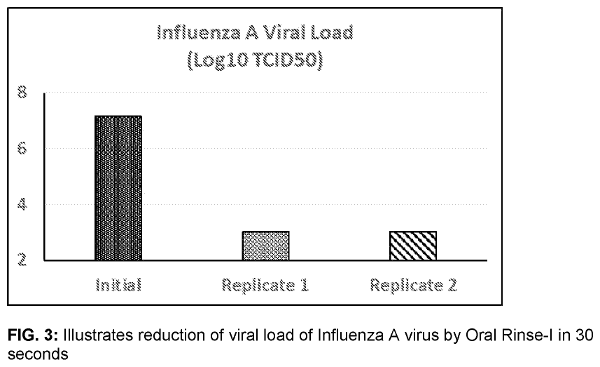

The results are summarized in Table 15 through 19 and in FIG. 3.

TABLE 15
Reduction of Influenza A virus by Oral Rinse-I

| | | Initial Viral | Output Viral | | |
|---|---|---|---|---|---|
| Contact | | Load | Load | Log₁₀ | % |
| Time | Replicate | (Log₁₀ TCID₅₀)* | (Log₁₀ TCID₅₀) | Reduction | Reduction |
| 30 Seconds | 1 | 7.16 | 3.03 | 4.13 | 99.993 |
| | 2 | 7.16 | 3.03 | 4.13 | 99.993 |
| 60 Seconds | 1 | 7.16 | 2.78 | 4.38 | 99.996 |
| | 2 | 7.16 | 2.53 | 4.63 | 99.998 |

*The average VRC for the corresponding contact time was used as initial viral load

TABLE 16
Reduction of Influenza A virus by Oral Rinse-II

| | | Initial Viral | Output Viral | | |
|---|---|---|---|---|---|
| Contact | | Load | Load | Log₁₀ | % |
| Time | Replicate | (Log₁₀ TCID₅₀)* | (Log₁₀ TCID₅₀) | Reduction | Reduction |
| 30 Seconds | 1 | 6.78 | 3.78 | 3.00 | 99.900 |
| | 2 | 6.78 | 3.03 | 3.75 | 99.982 |
| 60 Seconds | 1 | 6.78 | 2.78 | 4.00 | 99.990 |
| | 2 | 6.78 | 2.78 | 4.00 | 99.990 |

*The average VRC for the corresponding contact time was used as initial viral load

TABLE 17
Reduction of Influenza A virus by Oral Spray-X

| | | Initial Viral | Output Viral | | |
|---|---|---|---|---|---|
| Contact | | Load | Load | Log₁₀ | % |
| Time | Replicate | (Log₁₀ TCID₅₀)* | (Log₁₀ TCID₅₀) | Reduction | Reduction |
| 30 Seconds | 1 | 6.78 | 3.53 | 3.25 | 99.944 |
| | 2 | 6.78 | 3.28 | 3.50 | 99.968 |
| 60 Seconds | 1 | 6.78 | 3.28 | 3.50 | 99.968 |
| | 2 | 6.78 | 2.78 | 4.00 | 99.990 |

*The average VRC for the corresponding contact time was used as initial viral load

TABLE 18
Reduction of Influenza A virus by Toothpaste C*

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{**} | TCID₅₀) | TCID₅₀) | Reduction | Reduction |
| 30 | 1 | 6.95 | 4.53 | 2.42 | 2.58 | 99.73 |
| Seconds | 2 | | 4.28 | 2.67 | | |
| | 3 | | 4.28 | 2.67 | | |
| 60 | 1 | 6.95 | 4.03 | 2.92 | 2.83 | 99.84 |
| Seconds | 2 | | 4.28 | 2.67 | | |
| | 3 | | 4.03 | 2.92 | | |
| 120 | 1 | 6.95 | 3.78 | 3.17 | 3.25 | 99.94 |
| Seconds | 2 | | 3.78 | 3.17 | | |
| | 3 | | 3.53 | 3.42 | | |

*Supernatant of 1-3 diluted toothpaste slurry. The supernatant was treated as an undiluted sample.
^{**}The average VRC for the corresponding contact time was used as initial viral load

TABLE 19
Summary of Reduction of Influenza A virus by Oral Rinse-I,
Oral Rinse-II, Oral Spray-X and Toothpaste C

| | Reduction in Viral Load of Influenza A | | |
|---|---|---|---|
| Composition | 30 Seconds | 60 Seconds | 120 Seconds |
| Oral Rinse-I | 99.993%, 99.993% | 99.996%, 99.998% | Not Tested |
| Oral Rinse-II | 99.900%, 99.982% | 99.990%, 99.990% | Not Tested |
| Oral Spray-X | 99.944%, 99.968% | 99.968%, 99.990% | Not Tested |
| Toothpaste C* | 99.73% | 99.84% | 99.94% |

*Supernatant of 1 → 3 diluted toothpaste slurry. The supernatant was treated as an undiluted sample.

The results confirm that all controls were met affirming criteria for a valid test. The data showed that Oral Rinse I, Oral Rinse-II, Oral Spray-X and Toothpaste C reduced 99.993%, 99.9% to 99.982%, 99.944% to 99.968% and 99.73% of the initial viral load of the Influenza A virus within 30 seconds of contact, respectively. The reduction of viral load reduced the infectivity of Influenza A virus by 99.993%, 99.9% to 99.982%, 99.944% to 99.968% and 99.73% by Oral Rinse I, Oral Rinse-II, Oral Spray-X and Toothpaste C in 30 seconds, respectively. There was no significant substantive difference between 30- and 60-seconds of contact time for oral rinse and oral spray compositions. Similarly, there was no significant difference between 30-, 60- and 120-seconds of contact time for the toothpaste composition. As described herein, one of ordinary skill in the art would know that reduction in initial viral load of a virus is also referred to as the elimination of-, reducing the infectivity of-, reducing the viral count of-, destruction of- or killing of the virus. As described herein antiviral activity of Oral Rinse I, Oral Rinse-II, Oral Spray-X and Toothpaste C is thought to result from the oxidation of biomolecules, such as proteins and lipids of the Influenza A virus, by chlorine dioxide released from respective composition within 30 seconds or less of its contact with the virus. The formulation and dosage of stabilized source of chlorine dioxide is the same in Oral Rinse-I, Oral Rinse-II, Oral Rinse-III, Oral Spray-X, Oral Spray-Y, Toothpaste C, Toothpaste D and Toothpaste K. Therefore, similar antiviral activity against Influenza A virus is expected with Oral Rinse-III, Oral Spray-Y, Toothpaste D or Toothpaste K.

### EXAMPLE 8

### Antiviral Activity Against Human Coronavirus Strain 229E (HCoV-229E)

Antiviral activity of Oral Rinse-I against human coronavirus strain 229E was tested using a virucidal efficacy suspension test. The test method used was ASTM E1052 (see www.astm.org/Standards/E1052.htm) as recommended by ASTM International, formerly known as American Society for Testing Materials. This is a Standard Validated Test Method to Assess the Activity of Microbicides against Viruses in Suspension.

Test Conditions:
1. Test virus: Human coronavirus, strain 229E, ATCC VR-740.
2. Host: MRC-5 cells, ATCC CCL-171
3. Test Composition: Oral Rinse-I, as described in TABLE 2, above
4. Neutralization method: Serial 10-fold dilution using Eagle's Minimum Essential Medium (EMEM) supplemented with 2% fetal bovine serum (FBS)
5. Contact times: 30 and 60 seconds
6. Contact temperature: Room temperature (22.3-22.8° C. and 31% relative humidity)

Test Procedure:
1. Stock virus was thawed and was not supplemented with an organic soil load.
2. A 1:5 dilution of the stock virus was performed in sterile Ultrapure water.
3. Test and virus control substances were dispensed in 9-part equivalent volumes into sterile glass tubes.
4. Test and virus control substances were each inoculated with 1-part equivalent volumes of the test virus.
5. The test suspensions were held for 30 and 60 seconds of contact time and then neutralized by ten-fold serial dilutions into the appropriate solution.
6. The virus control suspension was neutralized in the same manner as the test suspensions.
7. Following neutralization, the viral suspensions were quantified to determine the levels of infectious virus using standard cell culture (e.g., TCID₅₀) assay techniques.
8. The cell culture plates were incubated for the period most suitable for the virus-host cell system (e.g., ˜7 days).
9. After the incubation period, the assay was scored for the presence/absence of test virus and cytotoxic effects. The appropriate calculations were performed (e.g., Spearman-Karber) to determine viral titers and levels of test substance cytotoxicity, where applicable.
10. Log₁₀ and percent reductions were computed for test suspensions relative to the control suspensions, and reported to the Study Sponsor.

Test Success Criteria:

The following measures were used to validate the virucidal efficacy data:
1. The virus titer control demonstrated obvious and or typical cytopathic effects on the monolayers unless a detection method other than cytopathic effect was used.
2. Neutralization of the test substance with a low titer (e.g., 1000-5000 infective units) of the test virus was demonstrated.
3. Quantification of the test and control parameters were conducted at a minimum of four determinations per dilution.

The product performance criteria follow:

The log and percent reduction of the test virus following exposure to the test substance were calculated however, there was no minimum reduction level to qualify as “passing” or an “efficacious” product.

Calculations

The TCID50 (Tissue Culture Infectivity Dose) represents the endpoint dilution where 50% of the cell cultures exhibit cytopathic effects due to infection by the test virus. The endpoint dilution at which 50% of the host cell monolayers exhibit cytotoxicity is termed the Tissue Culture Dose (TCD50). The TCID50 and TCD50 were determined using the Spearman-Karber method and calculated as follows:

Negative logarithm of endpoint titer=[−log of first dilution inoculated]−[((sum of % mortality at each dilution/100)-0.5)×Logarithm of dilution]

The result of this calculation is expressed as TCID50/0.1 ml (or volume of dilution inoculated) for the test, virus control, and neutralization control and TCD50/0.1 ml (or volume of dilution inoculated) for the cytotoxicity control.

Calculation of the Log₁₀Reduction: The log₁₀ reduction in viral titer was calculated as follows:

Plate Recovery Control Log₁₀ TCID50-Virus-Test Substance Logio TCID50

Calculation of the Percent Reduction: The percent reduction in viral titer was calculated as follows:

Percent Reduction=1−(CB)×100, where:

B=Average TCID₅₀ of virus in control suspensions.

C=Average TCID₅₀ of virus in virus-test suspensions.

The presence of any test substance cytotoxicity was taken into account when calculating the log and percent reductions in viral titer. If multiple virus control and test replicates were performed, the average TCID50 of each parameter was calculated and the average result used to calculate the log reductions in viral titer.

Results

The results were deemed acceptable and valid as (1) The Test Substance Neutralization Control demonstrated that the test substance was neutralized at <1.50 log₁₀ for the lot assayed and (2) no test substance cytotoxicity was detected in the lot of test substance assayed (<1.50 log₁₀).

The results for antiviral activity of Oral Rinse-I towards human coronavirus 229E are summarized in Table 20.

TABLE 20
Reduction of human coronavirus 229E by Oral Rinse-I

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{†} | TCID₅₀) | TCID₅₀) | Reduction | Reduction |
| 30 | 1 | 5.10 | 4.25 | 4.35 | 0.75 | 82.22 |
| Seconds | 2 | | 4.25 | | | |
| | 3 | | 4.50 | | | |
| 60 | 1 | 5.10 | 4.75 | 4.61 | 0.49 | 67.64 |
| Seconds | 2 | | 4.75 | | | |
| | 3 | | 4.00 | | | |

^{†}The mean viral recovery control value for the corresponding contact time was used as the Initial Load.

The results show that all controls were met affirming the validity of the test. The data show that Oral Rinse-I reduced 82.22% of the initial viral load of the human coronavirus 229E within 30 seconds. There was no substantiative difference in Logio reduction values between 30 seconds and 60 seconds of contact time; the 0.26 log difference between the contact times (0.75 vs. 0.49) is within the expected measurement variability of the test method used. Therefore, at room temperature, Oral Rinse-I was effective in reducing the viral load of human coronavirus 229E up to 82.22% within 30 seconds of contact time. The infectivity of human coronavirus 229E was thus reduced by 82.22% in 30 seconds. As described herein, one of ordinary skill in the art would know that reduction in initial viral load of a virus also refers to as the elimination of, reducing the infectivity of, reducing the viral count of, destruction of, or killing of the virus. Antiviral activity of Oral Rinse I is thought to result from the oxidation of biomolecules, such as proteins and lipids of the human coronavirus 229E, by chlorine dioxide released from Oral Rinse-I within seconds of its contact with the virus. The formulation and dosage of stabilized source of chlorine dioxide was the same in Oral Rinse-I, Oral Rinse-II, Oral Rinse-III, Oral Spray-X, Oral Spray-Y, Toothpaste C, Toothpaste D and Toothpaste K. Therefore, similar antiviral activity against human coronavirus 229E virus was anticipated for Oral Rinse-II, Oral Rinse-III, Oral Spray-X, Oral Spray-Y, Toothpaste C, Toothpaste D or Toothpaste K.

### EXAMPLE 9

### Antiviral Activity Against Rhinovirus Type 14, Adenovirus Type 5, Herpes Simplex Virus Type 1 and Herpes Simplex Virus Type 2.

Antiviral activity of Oral Rinse-I against Rhinovirus Type 14, Adenovirus Type 5, Herpes Simplex Virus Type 1 and Herpes Simplex Virus Type 2 was tested using a virucidal efficacy suspension test. The test method used was ASTM E1052-20 (see www.astm.org/Standards/E1052.htm) as recommended by ASTM International, formerly known as American Society for Testing Materials. This is a Standard Validated Test Method to Assess the Activity of Microbicides against Viruses in Suspension.

Test Conditions:
1. Rhinovirus Type 14:
   a. Test virus: Rhinovirus Type 14, Strain: 1059, Source: ATCC VR-284
   b. Host: H1-HeLa, Source: ATCC CRL-1958
2. Adenovirus Type 5:
   a. Test virus: Adenovirus Type 5, Strain: Adenoid 75, Source: ATCC VR-5
   b. Host: A549 cells, Source: ATCC CCL-185
3. Herpes Simplex Virus Type 1:
   a. Test virus: Herpes Simplex Virus Type 1, Strain: HF, Source: ATCC VR-260
   b. Host: Vero cells, Source: ATCC CCL-81
4. Herpes Simplex Virus Type 2:
   a. Test virus: Herpes Simplex Virus Type 2, Strain: G, Source: ATCC VR-734
   b. Host: Vero cells, Source: ATCC CCL-81
5. Test Composition: Oral Rinse-I as described in TABLE 2, above
6. Dilution medium: Minimum Essential Medium (MEM)+1.0 μg/mL Trypsin
7. Neutralizer: MEM+1% NCS+0.5% Sodium thiosulphate
8. Contact times: 30 and 60 seconds.
9. Contact temperature: 21° C.

Test Procedure: Test procedures for Rhinovirus Type 14, Adenovirus Type 5, Herpes Simplex Virus Type 1 and Herpes Simplex Virus Type 2 were same as described for Influenza A test in Example 7 of this specification except for the test virus and host cell.

Results:

The results are acceptable and valid as (1) virus was recovered from neutralizer effectiveness/viral interference (NE/VI) control, (2) viral-induced cytopathic effect (CPE) was distinguishable from test substance induced toxicity and (3) cell viability control remained viable throughout the course of the assay period in each of the test.

The results are summarized in Table 21 through 24.

TABLE 21
Reduction of Rhinovirus Type 14 by Oral Rinse-I

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{*} | TCID₅₀) | TCID₅₀) | Reduction | Reduction |
| 30 | 1 | 7.78 | 6.28 | 1.50 | 1.58 | 97.31 |
| Seconds | 2 | | 6.03 | 1.75 | | |
| | 3 | | 6.28 | 1.50 | | |
| 60 | 1 | 7.78 | 6.03 | 1.75 | 1.91 | 98.74 |
| Seconds | 2 | | 5.78 | 2.00 | | |
| | 3 | | 5.78 | 2.00 | | |

^{*}The average VRC for the corresponding contact time was used as initial viral load

TABLE 22
Reduction of Adenovirus Type 5 by Oral Rinse-I

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{*} | TCID₅₀) | TCID₅₀) | Reduction | Reduction |
| 30 | 1 | 6.95 | 5.28 | 1.67 | 1.75 | 98.18 |
| Seconds | 2 | | 5.28 | 1.67 | | |
| | 3 | | 5.03 | 1.92 | | |
| 60 | 1 | 6.95 | 4.78 | 2.17 | 2.08 | 99.15 |
| Seconds | 2 | | 4.78 | 2.17 | | |
| | 3 | | 5.03 | 1.92 | | |

^{*}The average VRC for the corresponding contact time was used as initial viral load

TABLE 23
Reduction of Herpes Simplex Virus Type 1 by Oral Rinse-I

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{*} | TCID₅₀) | TCID₅₀) | Reduction | Reduction |
| 30 | 1 | 8.28 | 5.53 | 2.75 | 2.66 | 99.77 |
| Seconds | 2 | | 5.53 | 2.75 | | |
| | 3 | | 5.78 | 2.50 | | |
| 60 | 1 | 8.28 | 5.28 | 3.00 | 2.91 | 99.87 |
| Seconds | 2 | | 5.28 | 3.00 | | |
| | 3 | | 5.53 | 2.75 | | |

^{*}The average VRC for the corresponding contact time was used as initial viral load

TABLE 24
Reduction of Herpes Simplex Virus Type 2 by Oral Rinse-I

| | | | | Mean | | |
|---|---|---|---|---|---|---|
| | | Initial | Output | Output | | |
| | | Load | Load | Load | Mean | |
| Contact | | (Log₁₀ | (Log₁₀ | (Log₁₀ | Log₁₀ | Mean % |
| Time | Replicate | TCID₅₀)^{*} | TCID₅₀) | TCID₅₀) | Reduction | Reduction |
| 30 | 1 | 8.11 | 5.03 | 3.08 | 3.24 | 99.94 |
| Seconds | 2 | | 4.78 | 3.33 | | |
| | 3 | | 4.78 | 3.33 | | |
| 60 | 1 | 8.11 | 4.78 | 3.33 | 3.41 | 99.96 |
| Seconds | 2 | | 4.78 | 3.33 | | |
| | 3 | | 4.53 | 3.58 | | |

^{*}The average VRC for the corresponding contact time was used as initial viral load

The results confirmed that all controls met the criteria for a valid test. Oral Rinse-I did not show any cytotoxicity to host cells. The data indicate that Oral Rinse-I reduced the initial viral load of Rhinovirus Type 14, Adenovirus Type 5, Herpes Simplex Virus Type 1 and Herpes Simplex Virus Type 2 by 97.3%, 98.18%, 99.77% and 99.94% within 30 seconds after first contact, respectively. Further exposure at 60 seconds did not increase the antiviral activity significantly. The infectivity of Rhinovirus Type 14, Adenovirus Type 5, Herpes Simplex Virus Type 1 and Herpes Simplex Virus Type 2 was thus reduced by 97.3%, 98.18%, 99.77% and 99.94% in 30 seconds, respectively. As described herein, one of ordinary skill in the art would know that reduction in initial viral load of a virus also refers to as the elimination of, reducing the infectivity of, reducing the viral count of, destruction of, or killing of the virus. Antiviral activity of Oral Rinse-I is thought to result from the oxidation of biomolecules, such as proteins and lipids of the viruses, by chlorine dioxide released from Oral Rinse-I within seconds of its contact with the virus. The formulation and dosage of stabilized source of chlorine dioxide was the same in Oral Rinse-I, Oral Rinse-II, Oral Rinse-III, Oral Spray-X, Oral Spray-Y, Toothpaste C, Toothpaste D and Toothpaste K. Therefore, similar antiviral activity against Rhinovirus Type 14, Adenovirus Type 5, Herpes Simplex Virus Type 1 and Herpes Simplex Virus Type 2 was anticipated for Oral Rinse-II, Oral Rinse-III, Oral Spray-X, Oral Spray-Y, Toothpaste C, Toothpaste D or Toothpaste K.

As described herein HCoV-229E causes common cold symptoms and is not highly pathogenic. Whereas, SARS-CoV-2 and SARS-CoV are highly contagious causing pandemic with high mortality rates. The data and results of testing the reduction of SARS-COV-2 (COVID-19 virus), SARS Co-V (SARS virus) and human coronavirus strain 229E as presented in Tables 6 through 11, Table 13 and Table 20, respectively demonstrate that each strain of human coronavirus responded differently to comparable treatments, affirming that the testing of the virucidal or virustatic effect of a composition on one strain of human coronavirus will not accurately predict the effect of the same composition and same method on another strain of human coronavirus. In that regard, it is unexpected to find such a large increase in efficacy i.e. reduction of ˜98.4%, ˜35% and 82.22% in 30 seconds of exposure to the same composition (Oral Rinse-I) between SARS-CoV-2, SARS-CoV and HCoV-229E.

As described herein, SARS-CoV-2, SARS-CoV and HCoV-229E are all members of human coronavirus family. All human coronaviruses have spike protein (S protein) as their structural characteristic, which is responsible for their infectivity via binding to the receptor. It is also known that three different antibodies against SARS-CoV do not bind successfully to the SARS-CoV-2 spike protein, further emphasizing structural differences between the spike proteins of these two infectious viruses. The S protein is comprised of 51 domain (or subunit) and S2 domain (or subunit) of which 51 domain binds to the ACE-2 receptor. Therefore, the structure of 51 domain appears to be important in determining the rate of infectivity of the virus. The primary structure of 51 domain of SARS-CoV-2 and SARS-CoV exhibit only 61% identity. Also, a section of 51 domain termed receptor-binding motif (RBM) affect the conformational flexibility of the protein thereby binding interactions with the ACE-2 receptor. The primary structure of RBM of SARS-CoV-2 and SARS-CoV exhibit only 50% identity. These structural differences have significant implications on pathogenesis, entry and ability to infect intermediate hosts for these coronaviruses. Moreover, these structural differences tend to suggest that different spike proteins exposed to the same oxidizing agent will react in differently. The changes in one or more of the tertiary and the quaternary structure of two different spike proteins that occur after exposure to an oxidizing agent may be different, potentially rendering one spike protein ineffective while perhaps only partially disabling the other spike protein.

Chlorine dioxide is an oxidizing agent and degrades proteins of viruses. A stabilized source of chlorine dioxide in oral rinse and toothpaste oxidizes salivary biomolecules including amnio acids such as L-methionine (U.S. patent application Ser. No. 15/605,506; US 2029/0070085 A1; Shewale J et al. Novel Dentifrices Exhibit Enhanced Effects Toward Achieving Good Oral Health; IADR/AADR/CADR General Session 98th General Session, Washington DC, Abstract # 2373; 2020). Data in Tables 7 and 13 demonstrate that Oral Rinse-I reduced the initial viral load of SARS-CoV-2 and SARS-CoV by 98.4% and 35.43% in 30 seconds of contact time. The viral load reduction methods described and taught herein measures reduction in infectivity of the virus to respective host cell. The spike protein structure, particularly RBM region of these two viruses responsible for their infectivity by binding to the ACE-2 receptor is significantly different (primary structure is only 50% identical). Therefore, the significant difference in the reduction of viral load within 30 seconds of contact time by Oral Rinse-I may be result of the difference in the extent of oxidation of amino acids in spike proteins of these viruses. Notably, a single oxidizing agent may have, at times, drastically different effects on two different viruses, and conversely, different oxidative compounds will affect the same virus differently.

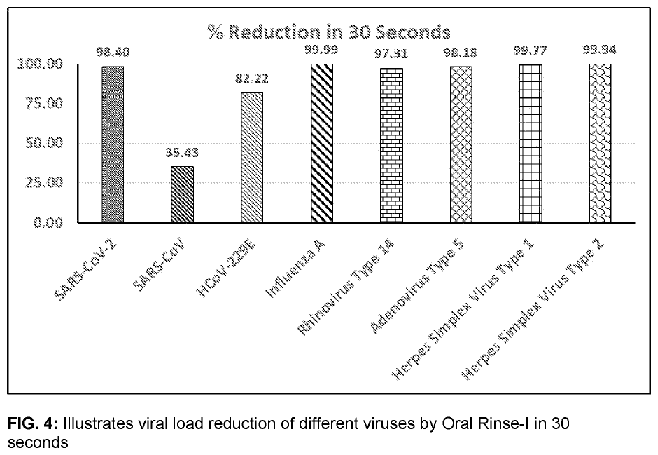

In partial summary of the above and with reference to FIG. 4, viral load reduction of different viruses by contact with Oral Rinse-I in 30 seconds is shown in accordance with various embodiments.

### EXAMPLE 10

### Antiviral Activity In Vivo

Antiviral activity of Oral Rinse-I will be studied in vivo to determine whether the use of Oral Rinse-I will be more effective at reducing SARS-CoV-2 viral load in the oral cavity than distilled water.

Participants in the study will comprise COVID-19 positive individuals. These individuals will complete questionnaires regarding demographics, socioeconomic status, comorbidities (e.g. asthma, diabetes, hypertension, body mass index), tobacco use, and COVID-19-related clinical symptoms. Participants will be instructed not to eat, drink, or smoke for at least 60 minutes before the sample collection process.

On the first day of the test (Day 1), participants will be asked to collect unstimulated saliva, wait for 10 minutes, and rinse their mouths/throats with 5m1 of provided distilled water, and collect the throat wash (gargle lavage) sample in a sterile container.

Participants will be instructed to use a provided DNA/RNA shield reagent in both saliva and throat collected samples to inactivate the virus for transportation which reduces inadvertent contamination. Next, the participants will be asked to rinse with 15mL of either Oral Rinse I or distilled water, collect unstimulated saliva after 5 minutes, and rinse again with a 5 mL distilled water bottle and collect the rinse liquid in another sterile container. Participants will then begin use of 15 mL of either Oral Rinse I or distilled water (control group) four times per day for 60 seconds. Participants may also have an oropharyngeal sample swab collected on Day 1.

Participants will continue to use of 15 mL of either Oral Rinse I or distilled water (control group) four times per day for 60 seconds on Days 2, 3, 4, 5, 6 and 7. On Day 7, participants will collect unstimulated saliva and throat wash samples in a manner similar to Day 1.

Participants will continue to use 15 mL of either Oral Rinse I or distilled water (control group) four times per day for 60 seconds on Days 7 through 28. On Day 28, participants will collect unstimulated saliva and throat wash samples in a manner similar to Day 1 and Day 7. Samples from Day 1, Day 7 and Day 28 will be analyzed. In particular, the samples will undergo a process to detect SARS-CoV-2 RNA using real-time reverse-transcription quantitative polymerase chain reaction (rRT-qPCR) to determine the viral load. Participants will be asked to answer follow up questions up to four times in the twelve months after Day 28.

Each of the exemplary compositions and those against which they were compared were suitable for use as a prophylactic treatment for the oral cavity or nasal channel and as routine home oral or nasal channel hygiene procedures.

In the above description, all cited references are incorporated herein by reference in their entireties. The citing of any reference is not an admission that such a reference is relevant prior art; rather, citations are to reference the novelty of the invention and discoveries described herein relative to known scientific literature, practices and prior art. In the description of the Present Invention, all ratios are weight ratios unless specifically stated otherwise. Unless otherwise indicated or evident from context, preferences indicated above and herein apply to the entirety of the embodiments discussed herein.

In describing the present invention, its embodiments and methods of use, the following terminology will be used: The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of items. The term “about” means quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide. Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3 and 4 and sub-ranges such as 1-3, 2-4 and 3-5, etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

It should be appreciated that the particular implementations shown and described herein are illustrative and are not intended to otherwise limit the scope of the present disclosure in any way. Furthermore, the connecting lines shown in the various figures contained herein are intended to represent exemplary functional relationships and/or physical couplings between the various elements. It should be noted that many alternative or additional functional relationships or physical connections may be present in a practical device or system.

It should be understood, however, that the detailed description and specific examples, while indicating exemplary embodiments, are given for purposes of illustration only and not of limitation. Many changes and modifications within the scope of the present disclosure may be made without departing from the spirit thereof, and the scope of this disclosure includes all such modifications. The corresponding structures, materials, acts, and equivalents of all elements in the claims below are intended to include any structure, material, or acts for performing the functions in combination with other claimed elements as specifically claimed. The scope should be determined by the appended claims and their legal equivalents, rather than by the examples given above. For example, the operations recited in any method claims may be executed in any order and are not limited to the order presented in the claims. Moreover, no element is essential unless specifically described herein as “critical” or “essential.”

Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

1. A method for reducing an initial viral load of Severe Acute Respiratory Syndrome Coronavirus-2 (SARS-CoV-2), comprising:
obtaining a single-phase multi-component antiviral composition comprising
from about 0.0005% to about 5.0% of at least one of sodium chlorite or potassium chlorite, based on a total weight of the single-phase multi-component antiviral composition,
a buffering system,
wherein pH of the single-phase multi-component antiviral composition is between 6.0 and 8.0, and water; and
contacting the single-phase multi-component antiviral composition with SARS-CoV-2 in a suspension for a contact time of at least 30 seconds, thereby reducing the initial viral load of SARS-CoV-2 by at least 98.4%.

2. The method of claim 1, wherein the contacting occurs in an oral cavity of a mammalian subject in need for reducing the viral load.

3. The method of claim 2, wherein the contacting comprises self-administration of the single-phase multi-component antiviral composition in the oral cavity, swishing for at least 30 seconds and spitting thereafter.

4. The method of claim 1, wherein the contacting occurs at room temperature.

5. The method of claim 1, wherein the obtaining further comprises obtaining the single-phase multi-component antiviral composition wherein the stabilized source of chlorine dioxide comprises sodium chlorite and wherein the buffering system comprises at least one of Na₃PO₄ and citric acid or Na₂HPO₄ and NaH₂PO₄.

6. The method of claim 5, wherein the obtaining further comprises obtaining the single-phase multi-component antiviral composition wherein the single-phase multi-component antiviral composition comprises a toothpaste comprising a N-acyl sarcosinate compound.

7. The method of claim 1, wherein the contacting comprises mixing the single-phase multi-component antiviral composition with the virus in a suspension.

8. The method of claim 2, wherein the contacting comprises administration of the single-phase multi-component antiviral composition.

9. The method of claim 1, wherein the contacting occurs in a throat of a mammalian subject in need for reducing the viral load.

10. The method of claim 9, wherein the contacting comprises self-administration of the single-phase multi-component antiviral composition in the throat, gargling for at least 30 seconds and spitting thereafter.

11. The method of claim 1, wherein the contacting occurs in a nasal cavity of a mammalian subject in need for reducing the viral load.

12. The method of claim 11, wherein the contacting comprises self-administration of the single-phase multi-component antiviral composition in at least one of a nasal spray or nasal rinse formulation into the nasal cavity.

13. A method for reducing an initial viral load of Influenza A, comprising:
obtaining a single-phase multi-component antiviral composition comprising
from about 0.0005% to about 5.0% of at least one of sodium chlorite or potassium chlorite, based on a total weight of the single-phase multi-component antiviral composition,
a buffering system,
wherein pH of the single-phase multi-component antiviral composition is between 6.0 and 8.0, and
water; and
contacting the single-phase multi-component antiviral composition with Influenza A in a suspension for a contact time of at least 30 seconds, thereby reducing the initial viral load of Influenza A by at least 99.9%.

14. The method of claim 13, wherein the contacting occurs in an oral cavity of a mammalian subject in need for reducing the viral load.

15. The method of claim 14, wherein the contacting comprises self-administration of the single-phase multi-component antiviral composition in the oral cavity, swishing for at least 30 seconds and spitting thereafter.

16. The method of claim 13, wherein the contacting occurs at room temperature.

17. The method of claim 13, wherein the obtaining further comprises obtaining the single-phase multi-component antiviral composition wherein the stabilized source of chlorine dioxide comprises sodium chlorite and wherein the buffering system comprises at least one of Na₃PO₄ and citric acid or Na₂HPO₄ and NaH₂PO₄.

18. The method of claim 17, wherein the obtaining further comprises obtaining the single-phase multi-component antiviral composition wherein the single-phase multi-component antiviral composition comprises a toothpaste comprising a N-acyl sarcosinate compound.

19. The method of claim 13, wherein the contacting comprises mixing the single-phase multi-component antiviral composition with the virus in a suspension.

20. The method of claim 14, wherein the contacting comprises administration of the single-phase multi-component antiviral composition.

21. The method of claim 13, wherein the contacting occurs in a throat of a mammalian subject in need for reducing the viral load.

22. The method of claim 13, wherein the contacting comprises self-administration of the single-phase multi-component antiviral composition in the throat, gargling for at least 30 seconds and spitting thereafter.

23. The method of claim 13, wherein the contacting occurs in a nasal cavity of a mammalian subject in need for reducing the viral load.

24. The method of claim 23, wherein the contacting comprises self-administration of the single-phase multi-component antiviral composition in at least one of a nasal spray or nasal rinse formulation into the nasal cavity.

25. A method for reducing an initial viral load of a virus, the virus comprising at least one of Herpes Simplex Virus Type 1, Herpes Simplex Virus Type 2, Rhinovirus Type 14, Adenovirus Type 5 or human coronavirus strain 229E (HCoV-229E) comprising:
obtaining a single-phase multi-component antiviral composition comprising
from about 0.0005% to about 5.0% of at least one of sodium chlorite or potassium chlorite, based on a total weight of the single-phase multi-component antiviral composition,
a buffering system,
wherein pH of the single-phase multi-component antiviral composition is between 6.0 and 8.0, and water; and
contacting the single-phase multi-component antiviral composition with the said virus in suspension for a contact time of at least 30 seconds, thereby reducing the initial viral load of the said virus in the range from 82.2% to 99.9%.

26. The method of claim 25, wherein the contacting occurs in an oral cavity of a mammalian subject in need reducing the viral load of said virus.

27. The method of claim 26, wherein the contacting comprises self-administration of the single-phase multi-component antiviral composition in the oral cavity, swishing for at least 30 seconds and spitting thereafter.

28. The method of claim 25, wherein the contacting occurs at room temperature.

29. The method of claim 25, wherein the step of obtaining further comprises the obtaining the single-phase multi-component antiviral composition wherein the stabilized source of chlorine dioxide comprises sodium chlorite and wherein the buffering system comprises at least one of Na₃PO₄ and citric acid or Na₂HPO₄ and NaH₂PO₄.

30. The method of claim 25, wherein the obtaining further comprises obtaining the single-phase multi-component antiviral composition wherein the single-phase multi-component antiviral composition comprises a toothpaste comprising a N-acyl sarcosinate compound.

31. The method of claim 25, wherein the contacting comprises mixing the single-phase multi-component antiviral composition with the said virus in a suspension.

32. The method of claim 26, wherein the contacting comprises administration of the single-phase multi-component antiviral composition.

33. The method of claim 1, wherein the single-phase multi-component antiviral composition is in the form of a rinse, a solution, a spray, a paste or a gel.

34. The method of claim 13, wherein the single-phase multi-component antiviral composition is in the form of a rinse, a solution, a spray, a paste or a gel.

35. The method of claim 25, wherein the single-phase multi-component antiviral composition is in the form of a rinse, a solution, a spray, a paste or a gel.
